(19) United States
(12) Patent Application Publication
(10) Pub. No.: US 2023/0366030 A1
(43) Pub. Date: Nov. 16, 2023

(54) METHODS OF DETECTING AN INACTIVATING MUTATION OF PBRM1 IN MENINGIOMA

(71) Applicant: Foundation Medicine, Inc., Cambridge, MA (US)

(72) Inventor: Erik WILLIAMS, Boston, MA (US)

(73) Assignee: Foundation Medicine, Inc., Cambridge, MA (US)

(21) Appl. No.: 18/025,365

(22) PCT Filed: Sep. 8, 2021
(86) PCT No.: PCT/US2021/049495

§ 371 Date: Mar. 8, 2023

Related U.S. Application Data
(60) Provisional application No. 63/076,191, filed on Sep. 9, 2020.

(51) Int. Cl. C12Q 1/6886

(52) U.S. Cl. CPC C12Q 1/6886; C12Q 2600/156

(57) ABSTRACT

Provided herein are methods for detecting an inactivating mutation of polybromo 1 (PBRM1) in an individual having meningioma, as well as methods of diagnosis, prognosis and treatment of meningioma related thereto.

### CROSS-REFERENCE TO RELATED APPLICATIONS

This application claims the benefit of U.S. Provisional Application No. 63/076,191, filed Sep. 9, 2020, which is hereby incorporated by reference in its entirety.

### FIELD

The present application relates to methods for detecting an inactivating mutation of polybromo 1 (PBRM1) in an individual having meningioma, as well as methods of diagnosis, prognosis and treatment of meningioma related thereto.

### BACKGROUND

Papillary meningioma (PM) is a type of brain tumor that is a World Health Organization (WHO) grade III meningioma subtype defined histologically by a predominant perivascular pseudopapillary growth pattern (Louis, D. N. et al. Acta Neuropathol. 2016 131:6). A papillary growth pattern in meningiomas has been associated with brain invasion and aggressive clinical behavior (Pasquier, B. et al. Cancer 1986 58:2; Kros, J. M. et al. Acta Neurol Scand. 2000 102:3; Avninder, S. et al. Diagn Pathol. 2007 2:3; Hojo, H. & Abe, M. Am J Surg Pathol. 2001 25:7). The standard treatment of papillary meningioma (PM) is surgical resection followed by radiation. However, most patients develop recurrent disease, and metastatic disease is common, particularly to the lung (Kros, J. M. et al. Acta Neurol Scand. 2000 102:3; Wang, D. J. et al. Int J Clin Exp Pathol. 2013 6:5).

Major obstacles to the identification of genomic alterations associated with PM have included low incidence of the tumor, scarcity of tumor tissue available for genomic analyses, and the presence of artifactual pseudo-papillary features in some meningiomas, which thereby confound cohorts (Avninder, S. et al. Diagn Pathol. 2007 2:3). Accordingly, there exists a need in the art for genetic testing that allows for effective diagnosis and treatment of PM.

All references cited herein, including patent applications and publications, are incorporated by reference in their entirety.

### SUMMARY

To meet these and other needs, provided herein are methods for detecting an inactivating mutation of polybromo 1 (PBRM1) in an individual having meningioma, as well as methods of diagnosis, prognosis and treatment of meningioma related thereto.

In certain aspects, provided herein is a method of treating or delaying progression of meningioma in an individual, comprising subjecting the individual to a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof, wherein the individual has an inactivating mutation of PBRM1. In some embodiments, an inactivating mutation of PBRM1 has been detected in a sample (e.g., a meningioma sample) of the individual prior to subjecting the individual to the therapy. In some embodiments, the method further comprises, prior to subjecting the individual to the therapy, detecting an inactivating mutation of PBRM1 in a sample (e.g., a meningioma sample) of the individual.

In certain aspects, provided herein is a method of identifying an individual having meningioma who may benefit from a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof, the method comprising detecting an inactivating mutation of PBRM1 in a sample from the individual, wherein presence of the inactivating mutation of PBRM1 in the sample identifies the individual as one who may benefit from the therapy. In some embodiments, the method further comprises providing a report comprising one or more treatment options comprising the therapy to the individual a physician treating the individual. In some embodiments, the method further comprises subjecting the individual to the therapy.

In certain aspects, provided herein is a method of selecting a therapy for an individual having meningioma, the method comprising detecting an inactivating mutation of PBRM1 in a sample from the individual, wherein presence of the inactivating mutation of PBRM1 in the sample identifies the individual as one who may benefit from a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the method further comprises providing a report comprising one or more treatment options comprising the therapy to the individual a physician treating the individual. In some embodiments, the method further comprises subjecting the individual to the therapy.

In certain aspects, provided herein is a method of identifying one or more treatment options for an individual having meningioma, the method comprising: (a) detecting presence of an inactivating mutation of PBRM1 in a sample from the individual; and (b) generating a report comprising one or more treatment options identified for the individual based at least in part on the presence of the inactivating mutation of PBRM1 in the sample, wherein the one or more treatment options comprise a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, report further comprises a score that associates the one or more treatment options with a predicted outcome and/or response. In some embodiments, the method further comprises subjecting the individual to the therapy.

In some embodiments according to any one of the methods described above, the therapy comprises aggressive tumor resection, e.g., Simpson grade I, II or III resection.

In some embodiments according to any one of the methods described above, the therapy comprises an adjuvant therapy following a standard therapy. In some embodiments, the standard therapy is tumor resection. In some embodiments, the standard therapy comprises radiotherapy after tumor resection. In some embodiments, the adjuvant therapy comprises one or more therapies selected from the group consisting of a targeted therapy, a chemotherapy, an anti-angiogenic therapy, a radiotherapy, an anti-inflammatory therapy, and a cancer immunotherapy.

In some embodiments according to any one of the methods described above, the therapy comprises an anti-cancer agent. In some embodiments, the anti-cancer agent is selected from the group consisting of an anti-angiogenic agent, a microtubule-destabilizing agent, a chemotherapeutic agent, an anti-DNA repair agent, and an anti-inflammatory agent. In some embodiments, the anti-cancer agent is an anti-angiogenic agent. In some embodiments, the anti-angiogenic agent is selected from the group consisting of axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept. In some embodiments, the anti-cancer agent is a microtubule-destabilizing agent. In some embodiments, the microtubule-destabilizing agent is selected from the group consisting of vinblastine, vincristine, vinorelbine, vinflunine, cryptophycins, halichondrins, dolastatins, hemiasterlins, colchicine, combrestatins, 2-methoxyestradiol, E7010, ombrabulin, soblidotin, D-24851, pseudolaric acid B, and embellistatin.

In some embodiments according to any one of the methods described above, the therapy comprises a cancer immunotherapy. In some embodiments, the cancer immunotherapy comprises one or more immunotherapies selected from the group consisting of a checkpoint inhibitor, cancer vaccine, cell-based therapy, T cell receptor (TCR)-based therapy, adjuvant immunotherapy, cytokine immunotherapy, and oncolytic virus therapy. In some embodiments, the cancer immunotherapy comprises small molecule, nucleic acid, polypeptide, carbohydrate, toxin, cell-based, or binding agent therapeutic agent. In some embodiments, the cancer immunotherapy comprises a checkpoint inhibitor. In some embodiments, the checkpoint inhibitor targets PD-L1, PD-1, CTLA-4, CEACAM, LAIR1, CD160, 2B4, CD80, CD86, CD276, VTCN1, HVEM, KIR, A2AR, MHC class I, MHC class II, GALS, adenosine, TGFR, OX40, CD137, CD40, IDO, CSF1R, TIM-3, BTLA, VISTA, LAG-3, TIGIT, IDO, MICA/B, or arginase.

In some embodiments according to any one of the methods described above, the therapy comprises a cancer immunotherapy comprising an agent that inhibits PD-1. In some embodiments, the agent that inhibits PD-1 is a small molecule, a nucleic acid, a polypeptide, carbohydrate, a lipid, a metal, or a toxin. In some embodiments, the agent that inhibits PD-1 is a PD-1 binding antagonist. In some embodiments, the PD-1 binding antagonist is an antibody, antibody-drug conjugate, antibody fragment, or immunoadhesin. In some embodiments, the PD-1 binding antagonist is selected from the group consisting of pembrolizumab, nivolumab, cemiplimab, spartalizumab, genolimzumab, SHR1210, JS001, BGB-108, BGB-A317, IBI308, GLS-010, BMS-936558, BCD-100, REGN2810, MGA-012, BI754091, STI-A1110, INCSHR-1210, PF-06801591, TSR-042, AM0001, JNJ-63723283, and ENUM 244C8.

In some embodiments according to any one of the methods described above, the therapy comprises a cancer immunotherapy comprising an agent that inhibits PD-L1 and/or PD-L2. In some embodiments, the agent that inhibits PD-L1 and/or PD-L2 is a small molecule, a nucleic acid, a polypeptide, carbohydrate, a lipid, a metal, or a toxin. In some embodiments, the agent that inhibits PD-L1 is a PD-L1 binding antagonist. In some embodiments, the PD-L1 binding antagonist is an antibody, antibody-drug conjugate, antibody fragment, or immunoadhesin. In some embodiments, the PD-L1 binding antagonist is selected from the group consisting of atezolizumab, avelumab, durvalumab, KN035, CS1001, MDX-1105, LY3300054, STI-A1014, FAZ053, and CX-072.

In some embodiments according to any one of the methods described above, the therapy comprises a cancer immunotherapy comprising an agent that inhibits CTLA4. In some embodiments, the agent that inhibits CTLA4 is a small molecule, a nucleic acid, a polypeptide, carbohydrate, a lipid, a metal, or a toxin. In some embodiments, the agent that inhibits CTLA4 is an antibody, antibody-drug conjugate, antibody fragment, or immunoadhesin. In some embodiments, the agent that inhibits CTLA4 is selected from the group consisting of ipilimumab, APL-509, AGEN1884, and CS1002. In some embodiments, the cancer immunotherapy comprises a combination of two or more checkpoint inhibitors.

In some embodiments, there is provided a method of identifying an individual having meningioma who may benefit from a cancer immunotherapy, the method comprising detecting an inactivating mutation of PBRM1 in a sample from the individual, wherein presence of the inactivating mutation of PBRM1 in the sample identifies the individual as one who may benefit from the cancer immunotherapy. In some embodiments, the method further comprises providing a report comprising one or more treatment options comprising the cancer immunotherapy to the individual a physician treating the individual. In some embodiments, the method further comprises administering to the individual an effective amount of the cancer immunotherapy. In some embodiments, the cancer immunotherapy comprises one or more immunotherapies selected from the group consisting of a checkpoint inhibitor, cancer vaccine, cell-based therapy, T cell receptor (TCR)-based therapy, adjuvant immunotherapy, cytokine immunotherapy, and oncolytic virus therapy. In some embodiments, the cancer immunotherapy comprises small molecule, nucleic acid, polypeptide, carbohydrate, toxin, cell-based, or binding agent therapeutic agent. In some embodiments, the cancer immunotherapy comprises a checkpoint inhibitor. In some embodiments, the checkpoint inhibitor targets PD-L1, PD-1, CTLA-4, CEACAM, LAIR1, CD160, 2B4, CD80, CD86, CD276, VTCN1, HVEM, KIR, A2AR, MEC class I, MEC class II, GALS, adenosine, TGFR, OX40, CD137, CD40, IDO, CSF1R, TIM-3, BTLA, VISTA, LAG-3, TIGIT, IDO, MICA/B, or arginase. In some embodiments, the cancer immunotherapy comprising an agent that inhibits PD-1, e.g., a PD-1 binding antagonist selected from the group consisting of pembrolizumab, nivolumab, cemiplimab, spartalizumab, genolimzumab, SHR1210, JS001, BGB-108, BGB-A317, IBI308, GLS-010, BMS-936558, BCD-100, REGN2810, MGA-012, BI 754091, STI-A1110, INCSHR-1210, PF-06801591, TSR-042, AM0001, JNJ-63723283, and ENUM 244C8. In some embodiments, the cancer immunotherapy is an agent that inhibits PD-L1 and/or PD-L2, e.g., a PD-L1 binding antagonist selected from the group consisting of atezolizumab, avelumab, durvalumab, KN035, CS1001, MDX-1105, LY3300054, STI-A1014, FAZ053, and CX-072. In some embodiments, the cancer immunotherapy comprises an agent that inhibits CTLA4, e.g., a CTLA4 antagonist selected from the group consisting of ipilimumab, APL-509, AGEN1884, and CS1002. In some embodiments, the cancer immunotherapy comprises a combination of two or more checkpoint inhibitors.

In certain aspects, provided herein is a method of diagnosing an individual having malignant meningioma (e.g., papillary meningioma), comprising detecting an inactivating mutation of PBRM1 in a sample from the individual, wherein presence of an inactivating mutation of PBRM1 indicates that the individual is likely to have malignant meningioma (e.g., papillary meningioma. In some embodiments, the method further comprises providing a report comprising one or more treatment options comprising a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof to the individual a physician treating the individual. In some embodiments, the method further comprises subjecting the individual to the therapy.

In certain aspects, provided herein is a method of providing a prognosis for an individual having meningioma, the method comprising detecting an inactivating mutation of PBRM1 in a sample from the individual, wherein presence of an inactivating mutation of PBRM1 identifies the individual as having a high risk of recurrent or metastatic meningioma. In some embodiments, the method further comprises providing a report comprising one or more treatment options comprising a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof to the individual a physician treating the individual. In some embodiments, the method further comprises subjecting the individual to the therapy.

In some embodiments according to any one of the methods described above, the presence of the inactivating mutation of PBRM1 is detected in DNA or RNA from the sample. In some embodiments, the presence of the inactivating mutation of PBRM1 is detected by polymerase chain reaction (PCR), Sanger sequencing, next-generation sequencing (NGS), single nucleotide polymorphism (SNP) array, or fluorescence in situ hybridization (FISH). In some embodiments, the presence of the inactivating mutation of PBRM1 is detected in protein from the sample. In some embodiments, the presence of the inactivating mutation of PBRM1 is detected in protein by immunohistochemistry.

In some embodiments according to any one of the methods described above, the method further comprises sequencing a target nucleic acid in the sample to determine the sequence or copy number of PBRM1, thereby detecting presence or absence of an inactivating mutation of PBRM1 in the sample.

In certain aspects, provided herein is a method of detecting an inactivating mutation of PBRM1 in a sample from an individual having meningioma. In some embodiments, the method comprises sequencing a target nucleic acid in the sample to determine the sequence or copy number of PBRM1, thereby detecting presence or absence of an inactivating mutation of PBRM1 in the sample. In some embodiments, the method is used for monitoring the individual before, during or after receiving a therapy for meningioma.

In some embodiments according to any one of the methods described above, the method further comprises contacting a bait with the sample from the individual to capture a target nucleic acid comprising PBRM1. In some embodiments, the target nucleic acid is DNA or RNA. In some embodiments, the target nucleic acid is a cell-free nucleic acid, such as cell-free DNA (cfDNA) or cell-free RNA (cfRNA). In some embodiments, the target nucleic acid is genomic DNA. In some embodiments, the target nucleic acid is mRNA.

In some embodiments according to any one of the methods described above, the sequencing is next-generation sequencing (NGS). In some embodiments, the sequencing is whole exome sequencing (WES), targeted sequencing or whole genome sequencing (WGS).

In some embodiments according to any one of the methods described above, the inactivating mutation of PBRM1 is loss of a PBRM1 allele. In some embodiments, the inactivating mutation of PBRM1 also results in loss of a BRCA1 associated polynucleotide (BAP1) allele.

In some embodiments according to any one of the methods described above, the inactivating mutation of PBRM1 is biallelic. In some embodiments, the inactivating mutation of PBRM1 is monoallelic. In some embodiments, the inactivating mutation of PBRM1 is selected from the group consisting of deletions (e.g., intragenic deletions, frame-shifting deletions, or deletions in coding sequence), insertions (e.g., frame-shifting insertions), truncating mutations and splice site mutations. In some embodiments, the inactivating mutation of PBRM1 is selected from the group consisting of F732fs*13, R146*, A482fs*18, Q949fs*59, E1029fs*100, K1372*, S39fs*14, S652fs*13, L1565fs*31 and V964fs*18.

In some embodiments according to any one of the methods described above, the inactivating mutation of PBRM1 results in reduced expression level of PBRM1 protein. In some embodiments, the inactivating mutation of PBRM1 results in reduced activity of PBRM1 protein.

In some embodiments according to any one of the methods described above, the inactivating mutation of PBRM1 is a germline mutation. In some embodiments, the inactivating mutation of PBRM1 is a somatic mutation. In some embodiments, the inactivating mutation of PBRM1 is present in the meningioma of the individual.

In some embodiments according to any one of the methods described above, the method further comprises obtaining the sample from the individual, e.g., prior to, during or after subjecting the individual to the therapy (e.g., cancer immunotherapy).

In some embodiments according to any one of the methods described above, the sample is a whole blood, serum, plasma, bone marrow, cerebrospinal fluid (CSF), tumor, or tissue sample. In some embodiments, the sample is from amniotic fluid, blood, plasma, serum, semen, lymphatic fluid, cerebral spinal fluid, ocular fluid, urine, saliva, stool, mucus, sweat, blood, skin, hair, hair follicles, saliva, oral mucous, vaginal mucus, sweat, tears, epithelial tissues, urine, semen, seminal fluid, seminal plasma, prostatic fluid, Cowper's fluid, excreta, biopsy, ascites, cerebrospinal fluid, or lymph. In some embodiments, the sample is a biopsy or formalin-fixed paraffin-embedded (FFPE) sample. In some embodiments, the sample is a tumor sample. In some embodiments, the sample comprises tumor nucleic acids.

In some embodiments according to any one of the methods described above, the method further comprises detecting one or more epigenetic modifications to PBRM1 in the individual. In some embodiments, the one or more epigenetic modifications comprise methylation.

In some embodiments according to any one of the methods described above, the meningioma is not characterized by a high tumor mutational burden (TMB). In some embodiments, the method further comprises determining a tumor mutational burden (TMB) in the sample from the individual. In some embodiments, the meningioma has a tumor mutational burden (TMB) of about 10 mutations/Mb or less, such as about 6.5 mutations/Mb or less.

In some embodiments according to any one of the methods described above, the meningioma is not characterized by microsatellite instability (MSI). In some embodiments, the method further comprises determining microsatellite instability (MSI) in the sample from the individual.

In some embodiments according to any one of the methods described above, the method further comprises detecting one or more additional mutations in the meningioma or the sample. In some embodiments, the one or more additional mutations are in one or more genes selected from the group consisting of ABL1, BRAF, CDKN1A, EPHA3, FGFR4, IKZF1, MCL1, NKX2-1, PMS2, RNF43, TET2, ACVR1B, BRCA1, CDKN1B, EPHB1, FH, INPP4B, MDM2, NOTCH1, POLD1, ROS1, TGFBR2, AKT1, BRCA2, CDKN2A, EPHB4, FLCN, IRF2, MDM4, NOTCH2, POLE, RPTOR, TIPARP, AKT2, BRD4, CDKN2B, ERBB2, FLT1, IRF4, MED12, NOTCH3, PPARG, SDHA, TNFAIP3, AKT3, BRIP1, CDKN2C, ERBB3, FLT3, IRS2, MEF2B, NPM1, PPP2R1A, SDHB, TNFRSF14, ALK, BTG1, CEBPA, ERBB4, FOXL2, JAK1, MEN1, NRAS, PPP2R2A, SDHC, TP53, ALOX12B, BTG2, CHEK1, ERCC4, FUBP1, JAK2, MERTK, NT5C2, PRDM1, SDHD, TSC1, AMER1, BTK, CHEK2, ERG, GABRA6, JAK3, MET, NTRK1, PRKAR1A, SETD2, TSC2, APC, C11orf30, CIC, ERRF11, GATA3, JUN, MITF, NTRK2, PRKCI, SF3B1, TYRO3, AR, CALR, CREBBP, ESR1, GATA4, KDM5A, MKNK1, NTRK3, PTCH1, SGK1, U2AF1, ARAF, CARD11, CRKL, EZH2, GATA6, KDM5C, MLH1, P2RY8, PTEN, SMAD2, VEGFA, ARFRP1, CASP8, CSF1R, FAM46C, GID4, (C17orf39), KDM6A, MPL, PALB2, PTPN11, SMAD4, VHL, ARID1A, CBFB, CSF3R, FANCA, GNA11, KDR, MRE11A, PARK2, PTPRO, SMARCA4, WHSC1, ASXL1, CBL, CTCF, FANCC, GNA13, KEAP1, MSH2, PARP1, QKI, SMARCB1, WHSC1L1, ATM CCND1, CTNNA1, FANCG, GNAQ, KEL, MSH3, PARP2, RAC1, SMO, WT1, ATR, CCND2, CT1NNB1, FANCL, GNAS, KIT, MSH6, PARP3, RAD21, SNCAIP, XPO1, ATRX, CCND3, CUL3, FAS, GRM3, KLHL6, MST1R, PAX5, RAD51, SOCS1, XRCC2, AURKA, CCNE1, CUL4A, FBXW7, GSK3B, KMT2A, (MLL), MTAP, PBRM1, RAD51B, SOX2, ZNF217, AURKB, CD22, CXCR4, FGF10, H3F3A, KMT2D, (MLL2), MTOR, PDCD1, RAD51C, SOX9, ZNF703, AXIN1, CD274, CYP17A1, FGF12, HDAC1, KRAS, MUTYH, PDCD1LG2, RAD51D, SPEN, AXL, CD70, DAXX, FGF14, HGF, LTK, MYC, PDGFRA, RAD52, SPOP, BAP1, CD79A, DDR1, FGF19, HNF1A, LYN, MYCL, PDGFRB, RAD54L, SRC, BARD1, CD79B, DDR2, FGF23, HRAS, MAF, MYCN, PDK1, RAF1, STAG2, BCL2, CDC73, DIS3, FGF3, HSD3B1, MAP2K1, MYD88, PIK3C2B, RARA, STAT3, BCL2L1, CDH1, DNMT3A, FGF4, ID3, MAP2K2, NBN, PIK3C2G, RB1, STK11, BCL2L2, CDK12, DOT1L, FGF6, IDH1, MAP2K4, NF1, PIK3CA, RBM10, SUFU, BCL6, CDK4, EED, FGFR1, IDH2, MAP3K1, NF2, PIK3CB, REL, SYK, BCOR, CDK6, EGFR, FGFR2, IGF1R, MAP3K13, NFE2L2, PIK3R1, RET, TBX3, BCORL1, CDK8, EP300, FGFR3, IKBKE, MAPK1, NFKBIA, PIM1, RICTOR, TEK, BCR, CD74, ETV4, ETV5, ETV6, EWSR1, EZR, MYB, NUTM1, RSPO2, SDC4, SLC34A2, TERC, TERT, and TMPRSS2. In some embodiments, the one or more additional mutations are in one or more genes selected from the group consisting of VF2, TBX3, CDKN2A, CREBBP, BAP1, NF2, ASXL1, FBXW7, NOTCH1, PTEN, SETD2, VHL, HGF, and TP53. In some embodiments, the one or more additional mutations comprise a mutation in BABP1.

In some embodiments according to any one of the methods described above, the method further comprises assessing histologic features of a tumor sample from the individual. In some embodiments, the tumor does not have obvious papillary features. In some embodiments, the tumor is papillary or has papillary features. In some embodiments, the tumor is rhabdoid or has rhabdoid features. In some embodiments, the tumor has heterogeneous histologic features. In some embodiments, the meningioma is Grade I, Grade II or Grade III.

In some embodiments according to any one of the methods described above, the individual is a mammal, such as a human.

In some embodiments according to any one of the methods described above, the individual has received a prior therapy (e.g., 1, 2, 3, or more cycles of prior therapy) for meningioma. In some embodiments, the prior therapy is selected from the group consisting of surgery, a targeted therapy, a chemotherapy, an anti-angiogenic agent, a radiotherapy, an anti-inflammatory therapy, an anti-DNA repair therapy, a cancer immunotherapy, and combinations thereof.

In certain aspects, provided herein is a kit comprising a reagent for detecting an inactivating mutation of PBRM1 in a sample from an individual having meningioma. In some embodiments, the reagent is a nucleic acid that hybridizes to a target nucleic acid comprising PBRM1 in the sample. In some embodiments, the reagent is a bait for capturing the target nucleic acid. In some embodiments, the target nucleic acid comprises one or more mutations of PBRM1 selected from the group consisting of F732fs*13, R146*, A482fs*18, Q949fs*59, E1029fs*100, K1372*, S39fs*14, S652fs*13, L1565fs*31 and V964fs*18. In some embodiments, the reagent is a PCR primer set for amplifying the target nucleic acid. In some embodiments, the reagent is an antibody that specifically binds to a PBRM1 protein.

In certain aspects, provided herein is an adjuvant therapy, an anti-cancer agent (e.g., an anti-angiogenic agent, or a microtubule-destabilizing agent), or a cancer immunotherapy for use in a method of treating or delaying progression of meningioma, wherein the method comprises administering an effective amount of the adjuvant therapy, the anti-angiogenic agent, the microtubule-destabilizing agent, and/or the cancer immunotherapy to an individual, and wherein an inactivating mutation of PBRM1 has been detected in a sample obtained from the individual. Also provided are use of an adjuvant therapy, an anti-cancer agent (e.g., an anti-angiogenic agent, or a microtubule-destabilizing agent), and/or a cancer immunotherapy in the preparation of a medicament for treating or delaying progression of meningioma, wherein an inactivating mutation of PBRM1 has been detected in a sample obtained from the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 provides a schematic diagram of PBRM1 truncating mutations identified in meningioma cases. The PBRM1 protein sequence is shown from N- (NH2, left side of protein) to C-terminus (COOH, right side of protein). Protein domains are indicated as rounded rectangles, and mutations that truncate the protein sequence are indicated with red triangles. Further description of the mutations shown is provided in Table 1.

FIGS. 2A-2D show histopathologic features of PBRM1-mutant meningioma. FIG. 2A shows tumor cells arranged in a papillary pattern (hematoxylin and eosin stain, 100× magnification). FIG. 2B shows a higher power image showing fragmentation of tissue architecture with preservation of perivascular tumor cells with cytoplasm tapering towards a perivascular nuclear-free region (hematoxylin and eosin stain, 400× magnification). FIG. 2C shows a high power image of rhabdoid meningioma showing prominent rhabdoid cytoplasmic inclusions, as indicated with a blue arrow (hematoxylin and eosin stain, 400× magnification). FIG. 2D shows chordoid meningioma, featuring chords of cells with vacuolated cytoplasm in a mucoid matrix (hematoxylin and eosin stain, 400× magnification).

### DETAILED DESCRIPTION

The present disclosure relates generally to the detection an inactivating mutation of polybromo 1 (PBRM1) in a patient having meningioma, as well as therapeutic methods related thereto. The present disclosure demonstrates that inactivating mutations of polybromo 1 (PBRM1) are associated with papillary meningioma, including meningioma with papillary features. While meningioma is typically a slow-growing tumor that can be treated by surgical resection, papillary meningioma is associated with aggressive clinical behavior and poor clinical prognosis. Thus, timely diagnosis is critical to guide selection of suitable treatment options and improve clinical outcome of patients with papillary meningioma. However, current diagnosis of papillary meningioma relies solely on post-operative histopathological analysis, and meningioma may have heterogeneous histological features that confound diagnosis. The present application provides a genomically-defined biomarker of papillary meningioma, thereby enabling improved patient stratification and therapeutic treatments.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers. The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

The term “inactivating mutation of PBRM1” to any mutation in a PBRM1-related nucleic acid or protein that results in reduced expression and/or activity of the PBRM1 protein.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“midge”), P(0)R, P(0)OR′, CO or CH₂ (“formacetal”), in which each R or R′ is independently H or substituted or unsubstituted alkyl (1 -20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “detection” or “detecting” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to a therapy). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial follicular fluid, seminal fluid, amniotic fluid, milk, whole blood, plasma, serum, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies (e.g., antibody-based checkpoint inhibitors) are used to delay development of a disease or to slow the progression of a disease.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., papillary meningioma), development of one or more clinical factors, or recovery from the disease.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “aggressive tumor resection” refers to surgical removal of a tumor at an early diagnostic stage that typically does not require tumor resection, and/or removal of the tumor at a higher degree of completeness than the individual would typically be indicated for based on histological and/or imaging diagnosis for the tumor. For example, an aggressive tumor resection may involve removal of the entire tumor even if it involves significant structures or organs that would counter-indicate tumor resection, and/or removal of tumors at distant sites away from the primary tumor in combination with removal of the primary tumor in an attempt to remove all gross tumors. For meningioma, the Simpson Grade is used to describe the degree of surgical resection completeness. Simpson Grade I refers to complete removal including resection of underlying bone and associated dura. Simpson Grade II refers to complete removal and coagulation of dural attachment. Simpson Grade III refers to complete removal without resection of dura or coagulation. Simpson Grade IV refers to subtotal resection. Simpson Grade V refers to simple decompression with or without biopsy. “Aggressive tumor resection” when used in the context of meningioma may refer to tumor resection with a high Simpson Grade level, e.g., Grade I, II or III, which is intended to remove the tumor as completely at macroscopic scale as feasible given the condition of the patient.

As used herein, “administering” is meant a method of giving a dosage of a compound (e.g., an antagonist) or a pharmaceutical composition (e.g., a pharmaceutical composition including an antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., a neoantigen or EWSR1-WT1 gene fusion) described herein. In some embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments described herein are specifically embraced by the present invention and are disclosed herein just as if each and every combination was individually and explicitly disclosed. In addition, all subcombinations of the embodiments describing such variables are also specifically embraced by the present invention and are disclosed herein just as if each and every such sub-combination was individually and explicitly disclosed herein.

### III. Methods

In one aspect, provided herein are methods of detecting an inactivating mutation of polybromo 1 (PBRM1) in an individual having meningioma. In some embodiments, the methods comprise detecting presence of an inactivating mutation of PBRM1 in a sample from the individual. In some embodiments, the presence of an inactivating mutation of PBRM1 is detected in vitro. The methods of detection described herein are useful for treatment, diagnosis, and prognosis of malignant meningioma, such as papillary meningioma, and for monitoring a patient having meningioma before, during or after receiving a therapy.

In some embodiments, provided herein are methods of treating or delaying progression of meningioma in an individual, comprising subjecting the individual to a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof, wherein the individual has an inactivating mutation of PBRM1. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

In some embodiments, provided herein are methods of treating or delaying progression of meningioma in an individual provided that an inactivating mutation of PBRM1 has been detected in a sample (e.g., a meningioma sample) of the individual, comprising subjecting the individual to a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

In some embodiments, provided herein are methods of treating or delaying progression of meningioma in an individual, comprising: (a) detecting an inactivating mutation of PBRM1 in a sample of the individual; and (b) subjecting the individual to a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

In another aspect, provided herein are methods of identifying an individual having meningioma who may benefit from a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof, comprising detecting an inactivating mutation of PBRM1 in a sample from the individual, wherein presence of the inactivating mutation of PBRM1 in the sample identifies the individual as one who may benefit from the therapy. In some embodiments, the method further comprises subjecting the individual to the therapy. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

In another aspect, provided herein are methods of selecting a therapy for an individual having meningioma, comprising detecting an inactivating mutation of PBRM1 in a sample from the individual, wherein presence of the inactivating mutation of PBRM1 in the sample identifies the individual as one who may benefit from a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the therapy comprises aggressive tumor resection. In some embodiments, the method further comprises subjecting the individual to the therapy. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

In another aspect, provided herein are methods of identifying one or more treatment options for an individual having meningioma, comprising: (a) detecting presence of an inactivating mutation of PBRM1 in a sample from the individual; and (b) generating a report comprising one or more treatment options identified for the individual based at least in part on the presence of the inactivating mutation of PBRM1 in the sample, wherein the one or more treatment options comprise a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the method further comprises subjecting the individual to the therapy. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor. In some embodiments, the report further comprises a score that associates the one or more treatment options with a predicted outcome and/or response.

In some embodiments, there is provided a method of generating a personalized cancer treatment report for an individual, comprising determining whether the individual has an inactivating mutation of PBRM1, selecting one or more treatment options based on the presence or absence of an inactivating mutation of PBRM1 in the individual, and generating a personalized cancer treatment report that indicates the presence or absence of an inactivating mutation of PBRM1 in the individual and the treatment option(s). In some embodiments, the report further comprises a score that associates the one or more treatment options with a predicted outcome and/or response. In some embodiments, the one or more treatment options comprise a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof. In some embodiments, the method further comprises subjecting the individual to the therapy. In some embodiments, the therapy comprises aggressive tumor resection. In some embodiments, the therapy comprises adjuvant therapy, e.g., following standard therapy. In some embodiments, the therapy comprises an anti-cancer agent. In some embodiments, the therapy comprises an anti-angiogenic agent. In some embodiments, the therapy comprises a microtubule-destabilizing agent, e.g., vinca alkaloids or colchicines. In some embodiments, the therapy comprises a cancer immunotherapy, such as an immune checkpoint inhibitor.

The present disclosure provides, inter alia, prognostic and pharmacodynamic methods. In some embodiments, provided herein are methods of providing a prognosis for an individual having meningioma, the method comprising detecting an inactivating mutation of PBRM1 in a sample from the individual, wherein presence of an inactivating mutation of PBRM1 identifies the individual as having a high risk of recurrent or metastatic meningioma. In some embodiments, the methods may involve monitoring a response of a patient to a therapy such as aggressive tumor resection, an adjuvant therapy, an anti-cancer agent (e.g., an anti-angiogenic agent, or a microtubule-destabilizing agent), and/or a cancer immunotherapy. In some embodiments, the methods and assays provided herein may be used to determine whether an individual having a meningioma is likely to respond to a therapy selected from the group consisting of aggressive tumor resection, an adjuvant therapy, an anti-cancer agent (e.g., an anti-angiogenic agent, or a microtubule-destabilizing agent), a cancer immunotherapy, and combinations thereof, the method including detecting presence of an inactivating mutation of PBRM1, wherein the presence of an inactivating mutation of PBRM1 identifies the individual as one who is likely to respond to the therapy. In some embodiments, the method further comprises subjecting the individual to the therapy.

Any of the methods of the present disclosure may include selecting a therapy for the individual. In some embodiments, the therapy comprises (e.g., the therapy is) aggressive tumor resection. In some embodiments, the therapy comprises (e.g., the therapy is) adjuvant therapy, e.g., following standard therapy. In some embodiments, the therapy comprises (e.g., the therapy is) an anti-angiogenic agent. In some embodiments, the therapy comprises (e.g., the therapy is) a microtubule-destabilizing agent, e.g., vinca alkaloids or colchicines. In some embodiments, the therapy comprises (e.g., the therapy is) a cancer immunotherapy, such as an immune checkpoint inhibitor. In some embodiments, the method further comprises subjecting the individual to aggressive tumor resection (e.g., Simpson Grade I, II or III resection). In some embodiments, the method further comprises administering an effective amount of an adjuvant therapy to the individual following a standard therapy (e.g., surgery or surgery and radiotherapy). In some embodiments, the method further comprises administering an effective amount of an anti-angiogenic agent to the individual. In some embodiments, the method further comprises administering an effective amount of a microtubule-destabilizing agent to the individual. In some embodiments, the method further comprises administering an effective amount of a cancer immunotherapy to the individual. In some embodiments, the method may further include selecting the therapy and/or subjecting the individual to the therapy. In some embodiments, the therapy is selected and/or administered to the individual as soon as possible.

Exemplary therapies and their administration are described infra. In some embodiments, an inactivating mutation of PBRM1 has been detected in a sample from the meningioma of the individual prior to subjecting the individual to the therapy of the present disclosure. In some embodiments, the methods further comprise detecting an inactivating mutation of PBRM1 in a sample from an individual prior to subjecting the individual to the therapy of the present disclosure. In some embodiments, the methods further comprise obtaining the sample from an individual prior to subjecting the individual to the therapy of the present disclosure.

In some embodiments, the methods further comprise generating a report comprising one or more treatment options identified for an individual based at least in part on the presence of an inactivating mutation of PBRM1, e.g., in a sample obtained from the individual. In some embodiments, the report further comprises a score that associates the one or more treatment options with a predicted outcome and/or response. In some embodiments, the report is in electronic, web-based or paper format. In some embodiments, the report identifies the presence or absence of an inactivating mutation of PBRM1 in the individual. In some embodiments, the report further comprises information on prognosis, resistance, suggested treatment options, likelihood of effectiveness of a treatment option, and/or recommendation of a treatment option. In some embodiments, the method comprises providing the report to the individual or a physician treating the individual.

The methods described herein are related to treatment, diagnosis and prognosis of meningioma. Meningioma is typically diagnosed through neurological examination followed by an imaging test, including use of computerized tomography (CT) scan, or magnetic resonance imaging (MRI). However, diagnosis of subtypes of meningioma usually rely on histopathological analysis of meningioma biopsy obtained during surgery of the tumor. Diagnosis of meningioma and its subtypes can be difficult because meningioma is typically slow growing, and may have heterogeneous histological features.

According to the 2007 World Health Organization's (WHO) classifications, papillary meningioma is defined as a subtype of “malignant meningiomas” (WHO Grade III) which feature the presence of a perivascular pseudopapillary pattern of tumor cell growth, either entirely or more commonly in combination with other common histological components of meningiomas. Rhabdoid meningioma is another subtype of meningioma based on histologic features, which are characterized by sheets of loosely cohesive, plump cells with eccentric nuclei and glassy, eosinophilic inclusion-like cytoplasm. Some meningioma samples may have both papillary and rhabdoid histologic features. Some meningioma samples may lack histologic features that are readily recognizable by a histologist in order to classify the meningioma as either papillary meningioma or rhabdoid meningioma, i.e., the meningioma does not have obvious papillary features, and/or the meningioma does not have obvious rhabdoid features.

In some embodiments, the meningioma is papillary meningioma. In some embodiments, the meningioma is meningioma with papillary features. In some embodiments, the meningioma does not have obvious papillary features. In some embodiments, the meningioma has heterogeneous histologic features. In some embodiments, the meningioma is anaplastic meningioma. In some embodiments, the meningioma is rhabdoid meningioma. In some embodiments, the meningioma is rhabdoid meningioma with papillary features. In some embodiments, the meningioma is chordoid meningioma. In some embodiments, the methods further comprise assessing histologic features of a tumor sample from the individual.

Many subtypes of meningioma is known, including, for example, cavernous sinus meningioma, cerebellopontine angle meningioma, cerebral convexity meningioma, foramen magnum meningioma, intraorbital meningioma, intraventricular meningioma, olgfactory groove meningioma, parasagittal/falx meningioma, petrous ridge meningioma, posterior fossa meningioma, sphenoid meningioma, spinal meningioma, suprasellar meningioma, and tentorium meningioma. In some embodiments, the meningioma is World Health Organization (WHO) grade I meningioma, i.e., a benign meningioma, including meningiothelial, fibrous (fibroblastic), transitional (mixed), psammomatous, angiomatous, microcystic, secretory, lymphoplasmacyte-rich, or metaplastic meningioma. In some embodiments, the meningioma is WHO grade II meningioma, i.e., atypical meningioma, such as chordoid, clear cell, or atypical meningioma. In some embodiments, the meningioma is WHO grade III meningioma, i.e., malignant meningioma, including, e.g., papillary, rhabdoid, or anaplastic meningioma. In some embodiments, the meningioma is metastatic meningioma. In some embodiments, the meningioma is advanced-stage or high grade meningioma. In some embodiments, the meningioma is not characterized by a high tumor mutational burden (TMB). In some embodiments, the meningioma has a tumor mutational burden (TMB) of about 10 mutations/Mb or less, such as about 6.5 mutations/Mb or less. In some embodiments, the meningioma is not characterized by microsatellite instability (MSI).

The methods described herein comprises detection of inactivating mutations of PBRM1, including any one of the PBRM1 mutations described in subsection “A. PBRM1 mutations” below. In some embodiments, the inactivating mutation of PBRM1 is loss of a PBRM1 allele. In some embodiments, the inactivating mutation of PBRM1 also results in loss of a BRCA1 associated polynucleotide (BAP1) allele. In some embodiments, the inactivating mutation of PBRM1 is biallelic. In some embodiments, the inactivating mutation of PBRM1 is monoallelic. In some embodiments, the inactivating mutation of PBRM1 is selected from the group consisting of deletions (e.g., intragenic deletions, frame-shifting deletions, or deletions in coding sequence), insertions (e.g., frame-shifting insertions), truncating mutations and splice site mutations. In some embodiments, the inactivating mutation of PBRM1 is selected from the group consisting of F732fs*13, R146*, A482fs*18, Q949fs*59, E1029fs*100, K1372*, S39fs*14, S652fs*13, L1565fs*31 and V964fs*18. In some embodiments, the inactivating mutation of PBRM1 results in reduced expression level of PBRM1 protein. In some embodiments, the inactivating mutation of PBRM1 results in reduced activity of PBRM1 protein. In some embodiments, the inactivating mutation of PBRM1 is a germline mutation. In some embodiments, the inactivating mutation of PBRM1 is a somatic mutation. In some embodiments, the inactivating mutation of PBRM1 is present in the meningioma of the individual.

The inactivating mutations of PBRM1 may be detected using any suitable methods, such as those described in the subsection “B. Methods of detection” below. In some embodiments, the presence of the inactivating mutation of PBRM1 is detected in DNA or RNA from the sample. In some embodiments, the presence of the inactivating mutation of PBRM1 is detected by polymerase chain reaction (PCR), Sanger sequencing, next-generation sequencing (NGS), single nucleotide polymorphism (SNP) array, or fluorescence in situ hybridization (FISH). In some embodiments, the presence of the inactivating mutation of PBRM1 is detected in protein from the sample. In some embodiments, the presence of the inactivating mutation of PBRM1 is detected in protein by immunohistochemistry.

In some embodiments, the method further comprises contacting a bait with the sample from the individual to capture a target nucleic acid comprising PBRM1. In some embodiments, the target nucleic acid is DNA or RNA. In some embodiments, the target nucleic acid is a cell-free nucleic acid, such as cell-free DNA (cfDNA) or cell-free RNA (cfRNA). In some embodiments, the target nucleic acid is genomic DNA. In some embodiments, the target nucleic acid is mRNA.

In some embodiments, the method comprises sequencing a target nucleic acid in the sample to determine the sequence or copy number of PBRM1, thereby detecting presence or absence of an inactivating mutation of PBRM1 in the sample. In some embodiments, the sequencing is next-generation sequencing (NGS). In some embodiments, the sequencing is whole exome sequencing (WES), targeted sequencing or whole genome sequencing (WGS).

In some embodiments, the method further comprises detecting one or more epigenetic modifications to PBRM1 in the individual. In some embodiments, the one or more epigenetic modifications comprise methylation.

The inactivating PBRM1 mutation(s) are detected in a sample of the individual. In some embodiments, the sample is a whole blood, serum, plasma, bone marrow, cerebrospinal fluid (CSF), tumor, or tissue sample. In some embodiments, the sample is from amniotic fluid, blood, plasma, serum, semen, lymphatic fluid, cerebral spinal fluid, ocular fluid, urine, saliva, stool, mucus, sweat, blood, skin, hair, hair follicles, saliva, oral mucous, vaginal mucus, sweat, tears, epithelial tissues, urine, semen, seminal fluid, seminal plasma, prostatic fluid, Cowper's fluid, excreta, biopsy, ascites, cerebrospinal fluid, or lymph. In some embodiments, the sample is a biopsy or formalin-fixed paraffin-embedded (FFPE) sample. In some embodiments, the sample is a tumor sample. In some embodiments, the sample comprises tumor nucleic acids.

The methods described herein may further comprise detection of one or more additional biomarkers other than PBRM1, and/or one or more diagnostic or prognostic steps. For example, in some embodiments, the method further comprises determining a tumor mutational burden (TMB) in the sample from the individual. In some embodiments, the method further comprises determining microsatellite instability (MSI) in the sample from the individual.

In some embodiments, the method further comprises assessing histologic features of a tumor sample from the individual. In some embodiments, the tumor does not have obvious papillary features. In some embodiments, the tumor is papillary or has papillary features. In some embodiments, the tumor is rhabdoid or has rhabdoid features. In some embodiments, the tumor has heterogeneous histologic features. In some embodiments, the meningioma is Grade I, Grade II or Grade III.

The method steps described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### A. PBRM1 Mutations

The methods described herein relate to detection of inactivating mutations of PBRM1, which reduce the expression and/or activity of PBRM1 protein.

PBRM1 is a 37-exon gene residing on chromosome 3p21, adjacent to BAP1, separated by approximately 0.135 megabase. PBRM1 encodes the BAF180 protein, the chromatin targeting subunit of the PBAF chromatin remodeling complex (Varela, I. et al. Nature 2011 27:469). PBRM1 is a tumor suppressor gene, mutated in 40% of clear cell renal cell carcinoma (RCC), as well as a subset of papillary RCC and bladder carcinoma (Varela, I. et al. Nature 2011 27:469; Biegel, J. A. et al. Am J Med Genet Part C Semin Med Genet. 2014 166C:3). Mutations in PBRM1 are most often truncations and result in loss of protein expression. Previous studies have illustrated a significant increase in cell proliferation and cell migration after knockdown of PBRM1 (Wang, H. K. et al. PLoS One 2017 12:8). Recent work has also demonstrated that BAF180 is required for centromeric cohesion, and DNA damage in cells lacking PBRM1 results in dynamic chromosome instability (Miao, D. et al. Science 359:6377). It has been speculated that the latter results in the improved survival of a subset of patients with PBRM1-mutant clear cell RCC cohorts treated with programmed cell death 1 receptor (PD-1) inhibitors (Miao, D. et al. Science 359:6377).

The nucleic acid and amino acid sequences of wildtype PBRM1 are known in the art and readily available on public databases, such as the National Center for Biotechnology Information (NCBI). In some embodiments, the PBRM1 gene is a human PBRM1 gene, also known as protein polybromo-1, polybromo-1D, BRG1-associated factor 180 (BAF180) or PB1. An exemplary PBRM1 gene is represented by NCBI Gene ID No. 55193. Exemplary nucleic acid sequences of human PBRM1 include, for example, human PBRM1 transcript variant 1 cDNA sequence (NCBI Reference sequence: NM_018313.4), human PBRM1 transcript variant 2 cDNA sequence (NCBI Reference sequence: NM 181042.4), and mouse PBRM1 cDNA sequence (NCBI Reference sequence: NM 001081251.1). Also contemplated herein are RNA nucleic acid sequences corresponding to the PBRM1 cDNA sequences described herein, nucleic acid molecules encoding orthologues of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequences described herein, or a portion thereof. Exemplary amino acid sequences of PBRM1 protein include, for example, human PBRM1 variant 1 amino acid sequence (NCBI Reference sequence: NP_060783.3), human PBRM1 variant 2 amino acid sequence (NCBI Reference sequence: NP 851385.1), and mouse PBRM1 protein sequence (NP 001074720.1). Also contemplated herein are orthologues of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any PBRM1 proteins, or a portion thereof.

Exemplary mutations include, but are not limited to, nucleic acid mutations including single-base substitutions, multi-base substitutions, insertion mutations, deletion mutations, frameshift mutations, missense mutations, nonsense mutations, splice-site mutations, epigenetic modifications (e.g., methylation, phosphorylation, acetylation, ubiquitylation, sumoylation, histone acetylation, histone deacetylation, and the like), and combinations thereof. In some embodiments, the mutation is a “nonsynonymous mutation,” meaning that the mutation alters the amino acid sequence of PBRM1. Such mutations reduce or eliminate PBRM1 protein amounts and/or function by eliminating proper coding sequences required for proper PBRM1 protein translation and/or coding for PBRM1 proteins that are non-functional or have reduced function (e.g., deletion of enzymatic and/or structural domains, reduction in protein stability, alteration of sub-cellular localization, and the like). Mutations contemplated herein include germline mutations and somatic mutations, such as mutations in the meningioma. Both biallelic and monoallelic mutations are contemplated herein.

In some embodiments, the inactivating mutation of PBRM1 is a loss-of-function mutation in the PBRM1 gene. In some embodiments, the inactivating mutation of PBRM1 is a nonsense, frameshift, or splice-site mutation. Such mutations may lead to lack of PBRM1 expression in cells harboring such mutations.

In some embodiments, the inactivating mutation of PBRM1 is loss of a PBRM1 allele. In some embodiments, the inactivating mutation of PBRM1 is biallelic loss of PBRM1. In some embodiments, the inactivating mutation of PBRM1 is monoallelic loss of PBRM1. In some embodiments, the inactivating mutation of PBRM1 also results in loss of a BRCA1 associated polynucleotide (BAP1) allele.

In some embodiments, the inactivating mutation of PBRM1 is a mutation that results in truncation of the PBRM1 protein. In some embodiments, the inactivating mutation of PBRM1 is selected from the group consisting of F732fs*13, R146*, A482fs*18, Q949fs*59, E1029fs*100, K1372*, 539fs*14, S652fs*13, L1565fs*31 and V964fs*18. Other inactivating mutations of PBRM1 may also be applicable, for example, see the inactivating mutations of PBRM1 described in WO2018/132287, which is incorporated herein by reference in its entirety.

In some embodiments, the inactivating mutation of PBRM1 reduces expression level of PBRM1 protein by any one of at least 10%, 20%, 30%, 40%, 50%, 60%, 70%, 80%, 90%, 95%, or more. In some embodiments, the inactivating mutation of PBRM1 results in no expression of PBRM1 protein. Expression level of PBRM1 gene products can be determined using known methods in the art, for example, by quantitative polymerase chain reaction (qPCR) or RNA sequencing for measuring RNA levels, or by western blot for measuring protein levels.

In some embodiments, the inactivating mutation of PBRM1 reduces activity of PBRM1 protein by any one of at least 10%, 20%, 30%, 40%, 50%, 60%, 70%, 80%, 90%, 95%, or more. The activity of PBRM1 protein can be measured using activity assays known in the art, for example, by assessing binding to acetylated Lys-14 of histone H3 (H3K14ac).

In some embodiments, the methods of the present disclosure further comprises detecting one or more epigenetic modifications to PBRM1 gene of the individual. In some embodiments, the individual has one or more epigenetic modifications to PBRM1 gene. In some embodiments, the one or more epigenetic modifications comprise methylations, such as methylations to the promoter, enhancer, and/or coding regions of the PBRM1 gene. In some embodiments, the one or more epigenetic modifications comprise histone modification. The one or more epigenetic modifications to PBRM1 gene may contribute to altered (e.g., lower) expression and/or activity level of PBRM1 gene products.

In some embodiments, the methods of the present disclosure further comprise detecting one or more additional mutations in a sample, tumor, or cancer. In some embodiments, the additional mutation(s) are in one or more genes other than PBRM1. In some embodiments, the additional mutation(s) are in one or more genes selected from the group consisting of ABL1, BRAF, CDKN1A, EPHA3, FGFR4, IKZF1, MCL1, NKX2-1, PMS2, RNF43, TET2, ACVR1B, BRCA1, CDKN1B, EPHB1, FH, INPP4B, MDM2, NOTCH1, POLD1, ROS1, TGFBR2, AKT1, BRCA2, CDKN2A, EPHB4, FLCN, IRF2, MDM4, NOTCH2, POLE, RPTOR, TIPARP, AKT2, BRD4, CDKN2B, ERBB2, FLT1, IRF4, MED12, NOTCH3, PPARG, SDHA, TNFAIP3, AKT3, BRIP1, CDKN2C, ERBB3, FLT3, IRS2, MEF2B, NPM1, PPP2R1A, SDHB, TNFRSF14, ALK, BTG1, CEBPA, ERBB4, FOXL2, JAK1, MEN1, NRAS, PPP2R2A, SDHC, TP53, ALOX12B, BTG2, CHEK1, ERCC4, FUBP1, JAK2, MERTK, NT5C2, PRDM1, SDHD, TSC1, AMER1, BTK, CHEK2, ERG, GABRA6, JAK3, MET, NTRK1, PRKAR1A, SETD2, TSC2, APC, C11orf30, CIC, ERRFI1, GATA3, JUN, MITF, NTRK2, PRKCI, SF3B1, TYRO3, AR, CALR, CREBBP, ESR1, GATA4, KDMSA, MKNK1, NTRK3, PTCH1, SGK1, U2AF1, ARAF, CARD11, CRKL, EZH2, GATA6, KDMSC, MLH1, P2RY8, PIEN, SMAD2, VEGFA, ARFRP1, CASP8, CSF1R, FAM46C, GID4, (C17orf39), KDM6A, MPL, PALB2, PTPN11, SMAD4, VHL, ARID1A, CBFB, CSF3R, FANCA, GNA11, KDR, MRE11A, PARK2, PTPRO, SMARCA4, WHSC1, ASXL1, CBL, CTCF, FANCC, GNA13, KEAP1, MSH2, PARP1, QKI, SMARCB1, WHSC1L1, ATM, CCND1, CTNNA1, FANCG, GNAQ, KEL, MSH3, PARP2, RAC1, SMO, WT1, ATR, CCND2, CTNNB1, FANCL, GNAS, KIT, MSH6, PARP3, RAD21, SNCAIP, XPO1, ATRX, CCND3, CUL3, FAS, GRM3, KLHL6, MST1R, PAXS, RAD51, SOCS1, XRCC2, AURKA, CCNE1, CUL4A, FBXW7, GSK3B, KMT2A, (MLL), MTAP, PBRM1, RAD51B, SOX2, ZNF217, AURKB, CD22, CXCR4, FGF10, H3F3A, KMT2D, (MLL2), MTOR, PDCD1, RAD51C, SOX9, ZNF703, AXIN1, CD274, CYP17A1, FGF12, HDAC1, KRAS, MUTYH, PDCD1LG2, RAD51D, SPEN, AXL, CD70, DAXX, FGF14, HGF, LTK, MYC, PDGFRA, RAD52, SPOP, BAP1, CD79A, DDR1, FGF19, HNF1A, LYN, MYCL, PDGFRB, RAD54L, SRC, BARD1, CD79B, DDR2, FGF23, HRAS, MAF, MYCN, PDK1, RAF1, STAG2, BCL2, CDC73, DIS3, FGF3, HSD3B1, MAP2K1, MYD88, PIK3C2B, RARA, STAT3, BCL2L1, CDH1, DNMT3A, FGF4, ID3, MAP2K2, NBN, PIK3C2G, RB1, STK11, BCL2L2, CDK12, DOT1L, FGF6, IDH1, MAP2K4, NF1, PIK3CA, RBM10, SUFU, BCL6, CDK4, EED, FGFR1, IDH2, MAP3K1, NF2, PIK3CB, REL, SYK, BCOR, CDK6, EGFR, FGFR2, IGF1R, MAP3K13, NFE2L2, PIK3R1, RET, TBX3, BCORL1, CDK8, EP300, FGFR3, IKBKE, MAPK1, NFKBIA, PIM1, RICTOR, TEK, BCR, CD74, ETV4, ETV5, ETV6, EWSR1, EZR, MYB, NUTM1, RSPO2, SDC4, SLC34A2, TERC, TERT, and TMPRSS2.

In some embodiments, the one or more additional mutation(s) are in one or more genes selected from the group consisting of VF2, TBX3, CDKN2A, CREBBP, BAP1, NF2, ASXL1, FBXW7, NOTCH1, PTEN, SETD2, VHL, HGF, and TP53. In some embodiments, the one or more additional mutations comprises a mutation in BAP1. In some embodiments, the one or more additional mutations are in other genes encoding components of the SWI/SNF complex. For example, component of the BAF complex, such as SMARCB1, SMARCE1 and ARID1A, have been previously reported in aggressive meningiomas (Abedalthagafi, M. S. et al. Cancer Genet. 2015 208:6; Perry, A. et al. Mod Pathol. 2005 18:7; Smith, M. J. et al. Nat Genet. 2013 45:3). It is postulated that the mutations resulting in disruption of SWI/SNF chromatin remodeling complexes are present in at least 20% of all human cancers (Biegel, J. A. et al. Am J Med Genet Part C Semin Med Genet. 2014 166C:3).

### B. Methods for Detection

Certain aspects of the present disclosure relate to detection of an inactivating mutation of PBRM1 in a sample, e.g., a patient sample. In some embodiments, the inactivating mutation of PBRM1 is detected in vitro.

In some embodiments, the presence or absence of an inactivating mutation of PBRM1 is detected in DNA from the sample. In some embodiments, the presence or absence of an inactivating mutation of PBRM1 is detected in RNA from the sample. In some embodiments, the presence or absence of an inactivating mutation of PBRM1 is detected in DNA and RNA from the sample.

Various methods for detecting an inactivating mutation of PBRM1 from DNA and/or RNA are known in the art. For example, in some embodiments, an inactivating mutation of PBRM1 is detected from DNA using next-generation sequencing (NGS), polymerase chain reaction (PCR), Sanger sequencing, or fluorescence in situ hybridization (FISH). In some embodiments, an inactivating mutation of PBRM1 is detected from RNA using RNA-sequencing (RNA-seq), polymerase chain reaction (PCR), Sanger sequencing, or fluorescence in situ hybridization (FISH). In some embodiments, an inactivating mutation of PBRM1 is detected from RNA by first synthesizing cDNA, then using RNA-sequencing (RNA-seq), polymerase chain reaction (PCR), Sanger sequencing, or fluorescence in situ hybridization (FISH). In some embodiments, the detection targets a specific or predetermined inactivating mutations of PBRM1. In some embodiments, the detection provides the sequence of an inactivating mutation of PBRM1.

In some embodiments, an inactivating mutation of PBRM1 is detected using one or more oligonucleotides. In some embodiments, an inactivating mutation of PBRM1 is detected by PCR amplification of a DNA or RNA sequence encoding any one of the PBRM1 mutations described herein, e.g., using two or more oligonucleotides that hybridize with portions of the DNA or RNA (e.g., cDNA) sequence on opposite strands. In some embodiments, an inactivating mutation of PBRM1 is detected by sequencing DNA or RNA sequence encoding any one of the PBRM1 mutations described herein, e.g., using one or more oligonucleotides that hybridize with portions of the DNA or RNA (e.g., cDNA) sequence. In some embodiments, an inactivating mutation of PBRM1 is detected by hybridization (e.g., in situ hybridization) of a DNA or RNA oligonucleotide with a sequence encoding any one of the PBRM1 mutations described herein.

In some embodiments, the methods of the present disclosure further comprise determining a tumor mutational burden (TMB), e.g., from a sample of the present disclosure. “Tumor mutational burden” is a measure of the number of nonsynonymous mutations in the tumor exome. Tumors with high TMB express large numbers of abnormal proteins. The prevalence of somatic mutations is considerably variable between different types of tumor. For example, non-small cell lung cancer (NSCLC) is typically associated with a high mutation frequency of 0.1 to 100 mut/Mb.

Methods for assessing TMB status are known in the art. See, for example, WO2017/151524, incorporated herein by reference in its entirety. For example, in some embodiments, the TMB of a sample or tumor is assessed by polynucleotide sequencing, e.g., quantifying a number of mutations detected per amount of DNA or RNA sequenced. In some embodiments, a sample or tumor with high TMB comprises at least about 10 mutations/Mb, at least about 15 mutations/Mb, at least about 20 mutations/Mb, or at least about 25 mutations/Mb. In some embodiments, a sample or tumor with low TMB comprises less than about 15 mutations/Mb, less than about 10 mutations/Mb, less than about 8 mutations/Mb, or less than about 6 mutations/Mb. In some embodiments, a sample, tumor, or cancer of the present disclosure is not characterized by high TMB. In some embodiments, a sample, tumor, or cancer of the present disclosure is characterized by low TMB. In some embodiments, a sample, tumor, or cancer of the present disclosure is characterized by a TMB of less than about 6.5 mutations/Mb. It is appreciated that TMB can be assessed by various methodologies known in the art, and high and low TMB ranges may differ depending on assay used.

In some embodiments, the methods of the present disclosure further comprise determining microsatellite instability (MSI) status of a sample, tumor, or cancer of the present disclosure. Methods for assessing MSI status are known in the art. For example, in some embodiments, MSI status is determined from at least about 50 loci, at least about 60 loci, at least about 70 loci, at least about 80 loci, at least about 90 loci, at least about 100 loci, or about 114 loci. In some embodiments, MSI status is determined by principal component analysis (PCA), the results of which can be used to threshold MSI status. In some embodiments, a sample, tumor, or cancer of the present disclosure is characterized by MSI. In some embodiments, a sample, tumor, or cancer of the present disclosure is not characterized by MSI.

In some embodiments, a sample of the present disclosure is a blood sample (e.g., a whole blood, plasma, or serum sample). In some embodiments, the sample (e.g., blood sample) obtained from the patient is selected from the group consisting of a whole blood, plasma, serum, or a combination thereof. In some embodiments, the sample is an archival blood sample, a fresh blood sample, or a frozen blood sample. In some embodiments, a sample of the present disclosure is a bone marrow sample. In some embodiments, a sample of the present disclosure is a cerebrospinal fluid (CSF) sample. In some embodiments, a sample of the present disclosure is a tissue sample. In some embodiments, a sample of the present disclosure is from, or comprises, amniotic fluid, blood, plasma, serum, semen, lymphatic fluid, cerebral spinal fluid, ocular fluid, urine, saliva, stool, mucus, sweat, blood, skin, hair, hair follicles, saliva, oral mucous, vaginal mucus, sweat, tears, epithelial tissues, urine, semen, seminal fluid, seminal plasma, prostatic fluid, Cowper's fluid, excreta, biopsy, ascites, cerebrospinal fluid, and/or lymph. In some embodiments, the sample is a solid sample. In some embodiments, the sample is a liquid sample.

In some embodiments, a sample of the present disclosure is a tumor sample, e.g., a sample of the meningioma. In some embodiments, a sample of the present disclosure is a biopsy sample. In some embodiments, a sample of the present disclosure is a formalin-fixed paraffin-embedded (FFPE) sample.

In some embodiments, cell free nucleic acid (cfNA), such as cell-free DNA (cfDNA) and/or cell-free RNA (ctRNA) and/or circulating tumor DNA (ctDNA) is isolated from a sample of the present disclosure (e.g., a whole blood sample, a plasma sample, a serum sample, a CSF sample, or a combination thereof). in some embodiments, the amount of cfNA (e.g., cfDNA) isolated from the sample is at least about 5 ng (e.g., at least about 5 ng, at least about 1 0 ng, at least about 15 ng, at least about 20 ng, at least about 25 ng, at least about 30 ng, at least about 35 ng, at least about 40 ng, at least about 45 ng, at least about 50 ng, at least about 75 ng, at least about 1 00 ng, at least about 200 ng, at least about 300 ng, at least about 400 ng, or more). For example, in some embodiments, the amount of cfNA (e.g., cfDNA) isolated from the sample is at least about 20 ng of cfNA (e.g., cfDNA). In some embodiments, the amount of cfNA (e.g., cfDNA) isolated from the sample is, for example, from about 5 ng to about 100 ng. In some embodiments, the amount of cfNA (e.g., cfDNA) isolated from the sample is about 100 ng or more.

Any suitable sample volume may be used in any of the methods described herein. For example, in some instances, the sample (e.g., a whole blood sample, a plasma sample, a serum sample, a CSF sample, or a combination thereof) may have a volume of about 1 mL to about 50 mL. In some embodiments, the sample (e.g., a whole blood sample, a plasma sample, a serum sample, a CSF sample, or a combination thereof) has a volume of about 10 mL. For example, in some instances, a plasma sample has a volume of 10 mL.

In some embodiments, the sample comprises a surface area of greater than or equal to about 25 mm². In some embodiments, the sample comprises a volume of greater than or equal to about 1 mm³. In some embodiments, the sample comprises at least about 30,000 cells. In some embodiments, the sample comprises at least about 80% cells. In some embodiments, the sample comprises greater than or equal to about 20% tumor content. In some embodiments, at least about 50 ng of dsDNA is obtained from the sample.

In some embodiments of any of the methods described herein, the sample from the individual is obtained from the individual prior to subjecting the individual to a therapy, e.g., aggressive tumor resection, an adjuvant therapy, an anti-cancer agent (e.g., an anti-angiogenic agent, or a microtubule-destabilizing agent), and/or a cancer immunotherapy. In other words, the sample may be a baseline sample.

### Detection of Inactivating Mutations

The following illustrative methods can be used to identify the presence of an in activating mutation of PBRM1, including structural alterations in a PBRM1 nucleic acid and/or polypeptide, changes in copy number of PBRM1, and changes in expression levels of PBRM1.

In some embodiments, detection of the mutation involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241: 1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample.

In some embodiments, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA.

In some embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759).

In some embodiments, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 7 4: 5463, and next generation sequencing (NGS).

Other methods for detecting mutations in a biomarker gene include methods in which protection from cleavage agents is used to detect mismatched bases in RNA/RNA or RNA/DNA heteroduplexes (Myers et al. (1985) Science 230: 1242). In some embodiments, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells.

In some embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad Sci USA 86:2766; see also Cotton (1993) Mutat. Res. 285:125-144 and Hayashi (1992) Genet. Anal. Tech. Appl. 9:73-79).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324: 163; Saiki et al. (1989) Proc. Natl. Acad Sci. USA 86:6230). Such allele specific oligonucleotides are hybridized to PCR amplified target DNA or a number of different mutations when the oligonucleotides are attached to the hybridizing membrane and hybridized with labeled target DNA

Methods of evaluating the copy number of a biomarker nucleic acid are well known to those of skill in the art. The presence or absence of chromosomal gain or loss can be evaluated simply by a determination of copy number of the regions or markers identified herein.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In some embodiments, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect RNA can be used, such that higher or lower expression relative to an appropriate control (e.g., a non-amplified portion of the same or related cell tissue, organ, etc.) provides an estimate of the relative copy number of the target nucleic acid. An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In some embodiments, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA Where there are chromosomal deletions or multiplications, differences in the ratio of the signals from the two labels will be detected and the ratio will provide a measure of the copy number.

In some embodiments, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60: 5405-5409. The known nucleic acid sequence for the genes is sufficient to enable one of skill in the art to routinely select primers to amplify any portion of the gene. Fluorogenic quantitative PCR may also be used in the methods of the present application. In fluorogenic quantitative PCR, quantitation is based on amount of fluorescence signals, e.g., TaqMan and SYBR green.

Gene expression may be assessed by any of a wide variety of well-known methods for detecting expression of a transcribed molecule or protein. Non-limiting examples of such methods include immunological methods, protein purification methods, protein function or activity assays, nucleic acid hybridization methods, nucleic acid reverse transcription methods, and nucleic acid amplification methods.

In some embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. Many techniques are known in the state of the art for determining absolute and relative levels of gene expression, commonly used techniques suitable for use in the present application include Northern analysis, RNase protection assays (RPA), microarrays and PCR-based techniques, such as quantitative PCR and differential display PCR.

In situ hybridization visualization may also be employed, wherein a radioactively labeled anti sense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts.

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In some embodiments, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In some embodiments, the primers and probes hybridize specifically under stringent conditions to a DNA fragment having the nucleotide sequence corresponding to the marker.

The form of labeling of the probes may be any that is appropriate, such as the use of radioisotopes, for example, 32P and 35S. Labeling with radioisotopes may be achieved, whether the probe is synthesized chemically or biologically, by the use of suitably labeled bases. In some embodiments, the biological sample contains polypeptide molecules from the test subject. Alternatively, the biological sample can contain mRNA molecules from the test subject or genomic DNA molecules from the test subject.

In some embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

The activity or level of a biomarker protein can be detected and/or quantified by detecting or quantifying the expressed polypeptide. The polypeptide can be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to an immune checkpoint therapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10⁻⁶M, 10⁻⁷M, 10⁻⁸M, 10⁻⁹M, or less. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

Epigenetic modifications may be detected using known methods in the art. For example, bisulfite conversion of methylated DNA followed by sequencing (e.g., NGS or Sanger sequencing), microarray analysis, qPCR, or PCR; or DNA enzyme digestion of methylated DNA followed by qPCR, PCR, sequencing (e.g., NGS or Sanger sequencing), HPLC-UV, LC-MS/MS or ELISA assay. In a bisulfite conversion method, a DNA sample is treated with sodium bisulfite resulting in the deamination of unmethylated cytosine to uracil and allowing the distinction between cytosine and methylated cytosine. The DNA enzyme digestion method is based on the use of DNA endonucleases which do not cut methylated DNA. Digestion of specific DNA target sequences by these enzymes generates DNA fragments of different lengths which may be sequenced to determine the extent of methylation.

### Nucleic Acid Capturing Reagents

The methods described herein may one or more nucleic acid molecules suitable as probe, primer, bait or library member that includes, flanks, hybridizes to, and which are useful for detecting, or are otherwise based on, the of the present disclosure relate to detection of inactivating mutations of PBRM1 as described herein. In some embodiments, the probe, primer or bait molecule is an oligonucleotide that allows capture, detection or isolation of an inactivating mutation of PBRM1 in a sample.

In some embodiments, the nucleic acid molecule is a probe or primer that includes an oligonucleotide between about 5 and 25, e.g., between 10 and 20, or 10 and 15 nucleotides in length. In some embodiments, the nucleic acid molecule is a bait that includes an oligonucleotide between about 100 to 300 nucleotides, 130 and 230 nucleotides, or 150 and 200 nucleotides, in length.

In some embodiments, the nucleic acid molecule can be used to identify or capture, e.g., by hybridization, an inactivating mutation of PBRM1. For example, the nucleic acid molecule can be a probe, a primer, or a bait, for use in identifying or capturing, e.g., by hybridization, an inactivating mutation of PBRM1 described herein.

The probes or primers described herein can be used, for example, for FISH detection or PCR amplification. In some embodiments, wherein detection is based on PCR, amplification of an inactivating mutation of PBRM1, can be performed using a primer or a primer pair, e.g., for amplifying a mutant sequence described herein. In some embodiments, a pair of isolated oligonucleotide primers can amplify a region containing or adjacent to a mutation. In some embodiments, the nucleic acid molecules can be used to identify, e.g., by hybridization, an inactivating mutation of PBRM1.

The nucleic acid molecule can be detectably labeled with, e.g., a radiolabel, a fluorescent label, a bioluminescent label, a chemiluminescent label, an enzyme label, a binding pair label, or can include an affinity tag; a tag, or identifier (e.g., an adaptor, barcode or other sequence identifier).

Also provided herein are isolated nucleic acid molecules encoding one or more of the inactivating mutations of PBRM1 as described herein. The nucleic acid molecule can be single-stranded or double-stranded; in certain embodiments the nucleic acid molecule is double-stranded DNA. Isolated nucleic acid molecules also include nucleic acid molecules sufficient for use as hybridization probes or primers to identify nucleic acid molecules that contain an inactivating mutation of PBRM1, e.g., nucleic acid molecules suitable for use as PCR primers for the amplification or mutation of nucleic acid molecules.

Probes based on the sequence of a mutant nucleic acid molecule can be used to detect transcripts or genomic sequences corresponding to inactivating mutations of PBRM1 as described herein. The probe comprises a label group attached thereto, e.g., a radioisotope, a fluorescent compound, an enzyme, or an enzyme co-factor. Such probes can be used as part of a test kit for identifying cells or tissues which express a mutant protein, such as by measuring levels of a nucleic acid molecule encoding the protein in a sample of cells from a subject, e.g., detecting mRNA levels or determining whether a gene encoding the protein has been mutated or deleted.

Typically these probes are 12 to 20, e.g., 17 to 20 nucleotides in length (longer for large insertions) and have the nucleotide sequence corresponding to the region of the mutations at their respective nucleotide locations on the gene sequence. Such molecules can be labeled according to any technique known in the art, such as with radiolabels, fluorescent labels, enzymatic labels, sequence tags, biotin, other ligands, etc. As used herein, a probe that “specifically hybridizes” to a mutant gene sequence will hybridize under high stringency conditions.

A probe will typically contain one or more of the specific mutations described herein. Typically, a nucleic acid probe will encompass only one mutation. Such molecules may be labeled and can be used as allele-specific probes to detect the mutation of interest.

The term “primer” as used herein refers to a sequence comprising two or more deoxyribonucleotides or ribonucleotides, e.g., more than three, and more than eight, or at least 20 nucleotides of a sequence corresponding to a sequence flanking an inactivating mutation of PBRM1 as described herein. Primers may be used to initiate DNA synthesis via the PCR (polymerase chain reaction) or a sequencing method, e.g., by an NGS method. The primers can specifically hybridize, for example, to the ends of the exons or to the introns flanking the exons. The amplified segment can then be further analyzed for the presence of the mutation such as by a sequencing method, or by a size separation technique such as by electrophoresis on a gel. The primers are useful in directing amplification of a target region that includes a mutation, e.g., prior to sequencing.

A primer is typically single stranded, e.g., for use in sequencing or amplification methods, but may be double stranded. If double stranded, the primer may first be treated to separate its strands before being used to prepare extension products. A primer must be sufficiently long to prime the synthesis of extension products in the presence of the inducing agent for polymerization. The exact length of primer will depend on many factors, including applications (e.g., amplification method), temperature, buffer, and nucleotide composition. A primer typically contains 12-20 or more nucleotides.

Primers are typically designed to be “substantially” complementary to each strand of a genomic locus to be amplified. Thus, the primers must be sufficiently complementary to specifically hybridize with their respective strands under conditions which allow the agent for polymerization to perform. In other words, the primers should have sufficient complementarity with the 5′ and 3′ sequences flanking the mutation to hybridize therewith and permit amplification of the genomic locus. The term “substantially complementary to” or “substantially the sequence” refers to sequences that hybridize to the sequences provided under stringent conditions and/or sequences having sufficient homology with a sequence comprising a mutation, e.g., a point mutation, or the wildtype counterpart sequence, such that the allele specific oligonucleotides hybridize to the sequence.

In some embodiments, the nucleic acid molecule is a bait. A bait can be a nucleic acid molecule, e.g., a DNA or RNA molecule, which can hybridize to (e.g., be complementary to), and thereby allow capture of a target nucleic acid that corresponds to any one of the inactivating mutations of PBRM1 described herein. In certain embodiments, the target nucleic acid is a genomic DNA molecule. In some embodiments, the target nucleic acid is an RNA molecule or a cDNA molecule derived from an RNA molecule. In some embodiments, a bait is an RNA molecule. In some embodiments, a bait includes a binding entity, e.g., an affinity tag, that allows capture and separation, e.g., by binding to a binding entity, of a hybrid formed by a bait and a nucleic acid hybridized to the bait. In some embodiments, a bait is suitable for solution phase hybridization. Typically, RNA molecules are used as bait sequences. A RNA-DNA duplex is more stable than a DNA-DNA duplex, and therefore provides for potentially better capture of nucleic acids.

RNA baits can be made using methods known in the art including, but not limited to, de nova chemical synthesis and transcription of DNA molecules using a DNA-dependent RNA polymerase. In some embodiments, the bait sequence is produced using known nucleic acid amplification methods, such as PCR, e.g., using human DNA or pooled human DNA samples as the template. The oligonucleotides can then be converted to RNA baits. In some embodiments, in vitro transcription is used, for example, based on adding an RNA polymerase promoter sequence to one end of the oligonucleotide. In some embodiments, the RNA polymerase promoter sequence is added at the end of the bait by amplifying or reamplifying the bait sequence, e.g., using PCR or other nucleic acid amplification methods, e.g., by tailing one primer of each target-specific primer pairs with an RNA promoter sequence. In some embodiments, the RNA polymerase is a T7 polymerase, a SP6 polymerase, or a T3 polymerase. In some embodiments, RNA bait is labeled with a tag, e.g., an affinity tag. In one embodiment, RNA bait is made by in vitro transcription, e.g., using biotinylated UTP. In another embodiment, RNA bait is produced without biotin and then biotin is crosslinked to the RNA molecule using methods well known in the art, such as psoralen crosslinking. In some embodiments, the RNA bait is an RNase-resistant RNA molecule, which can be made, e.g., by using modified nucleotides during transcription to produce a RNA molecule that resists RNase degradation. In some embodiments, the RNA bait corresponds to only one strand of the double-stranded DNA target. Typically, such RNA baits are not self-complementary and are more effective as hybridization drivers.

The bait sets can be designed from reference sequences, such that the baits are optimal for selecting targets of the reference sequences. In some embodiments, bait sequences are designed using a mixed base (e.g., degeneracy). For example, the mixed base(s) can be included in the bait sequence at the position(s) of a common SNP or mutation, to optimize the bait sequences to catch both alleles (e.g., SNP and non-SNP; mutant and non-mutant). In some embodiments, all known sequence variations (or a subset thereof) can be targeted with multiple oligonucleotide baits, rather than by using mixed degenerate oligonucleotides.

In certain embodiments, the bait set includes an oligonucleotide (or a plurality of oligonucleotides) between about 100 nucleotides and 300 nucleotides in length. Typically, the bait set includes an oligonucleotide (or a plurality of oligonucleotides) between about 130 nucleotides and 230 nucleotides, or about 150 and 200 nucleotides, in length. In some embodiments, the bait set includes an oligonucleotide (or a plurality of oligonucleotides) between about 300 nucleotides and 1000 nucleotides in length. In some embodiments, the target member-specific sequences in the oligonucleotide are between about 40 and 1000 nucleotides, about 70 and 300 nucleotides, about 100 and 200 nucleotides in length, typically between about 120 and 170 nucleotides in length.

In some embodiments, the bait set includes a binding entity. The binding entity can be an affinity tag on each bait sequence. In some embodiments, the affinity tag is a biotin molecule or a hapten. In certain embodiments, the binding entity allows for separation of the bait/member hybrids from the hybridization mixture by binding to a partner, such as an avidin molecule, or an antibody that binds to the hapten or an antigen-binding fragment thereof.

In some embodiments, the oligonucleotides in the bait set contain forward and reverse complement sequences for the same target member sequence whereby the oligonucleotides with reverse complemented member-specific sequences also carry reverse complement universal tails. This can lead to RNA transcripts that are the same strand, i.e., not complementary to each other.

In some embodiments, the bait set includes oligonucleotides that contain degenerate o mixed bases at one or more positions. In still other embodiments, the bait set includes multiple or substantially all known sequence variants present in a population of a single species or community of organisms. In some embodiments, the bait set includes multiple or substantially all known sequence variants present in a human population.

In some embodiments, the bait set includes cDNA sequences or is derived from cDNA sequences. In some embodiments, the bait set includes amplification products (e.g., PCR products) that arc amplified from genomic DNA, cDNA or cloned DNA.

In some embodiments, the bait set includes RNA molecules. In some embodiments, the set includes chemically, enzymatically modified, or in vitro transcribed RNA molecules, including but not limited to those that are more stable and resistant to RNase.

In yet other embodiments, the baits are produced by methods described in US 2010/0029498 and Gnirke, A. et al. (2009) Nat Biotechnol. 27(2):182-189, incorporated herein by reference. For example, biotinylated RNA baits can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In some embodiments, libraries of synthetic oligodeoxynucleotides can be obtained from commercial suppliers, such as Agilent Technologies, Inc., and amplified using known nucleic acid amplification methods.

The bait sequences described herein can be used for selection of exons and short target sequences. The target-specific sequences in the baits, e.g., for selection of exons and short target sequences, are between about 40 nucleotides and 1000 nucleotides in length. In one embodiment, the target-specific sequence is between about 70 nucleotides and 300 nucleotides in length. In another embodiment, the target-specific sequence is between about 100 nucleotides and 200 nucleotides in length. In yet another embodiment, the target-specific sequence is between about 120 nucleotides and 170 nucleotides in length. In some embodiments, long oligonucleotides can minimize the number of oligonucleotides necessary to capture the target sequences. For example, one oligonucleotide can be used per exon.

Also provided herein are a library of baits for capturing nucleic acid molecules corresponding to any one of the inactivating mutations of PBRM1 described herein and optionally one or more additional mutations described herein.

### Sequencing

The methods described herein may include one or more sequencing steps, e.g., by NGS sequencing. Any of a variety of sequencing reactions known in the art can be used to directly sequence at least a portion of a mutant gene. In some embodiments, the mutant gene sequence is compared to a corresponding reference (control) sequence.

Any method of sequencing known in the art can be used. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). Any of a variety of automated sequencing procedures can be utilized when performing the assays (Biotechniques (1995) 19:448), including sequencing by mass spectrometry (see, for example, U.S. Pat. No. 5,547,835 and international patent application Publication Number WO94/16101, entitled DNA Sequencing by Mass Spectrometry by H. Koster; U.S. Pat. No. 5,547,835 and international patent application Publication Number WO 94/21822 entitled DNA Sequencing by Mass Spectrometry Via Exonuclease Degradation by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159).

Sequencing of nucleic acid molecules can also be carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules are sequenced simultaneously). In some embodiments, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference.

Platforms for next-generation sequencing include, but are not limited to, Illumina NOVASEQ™ 6000 Sequencing System, Illumina HISEQ™ platforms, Illumina MISEQ™ platforms, PacBio SEQUEL® Systems, 10× Genomics CHROMIUM™ Controller, NanoString GEOIVIX™ Digital Spatial Profiler, and Ion Torrent platforms.

In some embodiments, the NGS is whole genome sequencing (WGS), which determines the sequences of the entire genome. In some embodiments, the NGS is targeted sequencing. Targeted sequencing focuses on specific areas of the genome. Exemplary methods of targeted NGS include hybridization capture and amplicon sequencing. For example, genomic regions of interest may be enriched by hybridizing genomic DNA sample to target-specific oligonucleotides, e.g., biotinylated oligos, which may be subsequently separated from the non-hybridized DNA in the sample and subject to sequencing analysis. In some embodiments, the NGS is whole exome sequencing (WES). Whole exome sequencing is a targeted NGS method that identifies all the protein-coding genes (i.e., exons) in the genome. NGS technologies can include one or more of steps, e.g., template preparation, sequencing and imaging, and data analysis.

In some embodiments, the method comprises isolating a nucleic acid sample to provide a library. In some embodiments, the nucleic acid sample includes whole genomic, subgenomic fragments, or both. Protocols for isolating and preparing libraries from whole genomic or subgenomic fragments are known in the art (e.g., Illumina's genomic DNA sample preparation kit). In certain embodiments, the genomic or subgenomic DNA fragment is isolated from a subject's sample (e.g., a tumor sample, a normal adjacent tissue (NAT), a blood sample or any normal control)).

In some embodiments, the nucleic acid sample used to generate the library includes RNA or cDNA derived from RNA. In some embodiments, the RNA includes total cellular RNA. In some embodiments, certain abundant RNA sequences (e.g., ribosomal RNAs) have been depleted. In some embodiments, the poly(A)-tailed mRNA fraction in the total RNA preparation has been enriched. In some embodiments, the cDNA is produced by random-primed cDNA synthesis methods. In some embodiments, the cDNA synthesis is initiated at the poly(A) tail of mature mRNAs by priming by oligo(dT)-containing oligonucleotides.

In some embodiments, the nucleic acid sample is fragmented or sheared by physical or enzymatic methods and ligated to synthetic adapters, size-selected (e.g., by preparative gel electrophoresis) and amplified (e.g., by PCR). In some embodiments, the fragmented and adapter-ligated group of nucleic acids is used without explicit size selection or amplification prior to hybrid selection.

In some embodiments, the isolated DNA (e.g., the genomic DNA) is fragmented or sheared. In some embodiments, the library includes less than 50% of genomic DNA, such as a subfraction of genomic DNA that is a reduced representation or a defined portion of a genome, e.g., that has been subfractionated by other means. In some embodiments, the library includes all or substantially all genomic DNA.

The method can further include amplifying the nucleic acid sample by specific or non-specific nucleic acid amplification methods that are well known to those skilled in the art. In some embodiments, the nucleic acid sample is amplified, e.g., by whole-genome amplification methods such as random-primed strand-displacement amplification. Template amplification methods such as PCR can be coupled with NGS platforms to target or enrich specific regions of the genome (e.g., exons). Exemplary template enrichment methods include, e.g., microdroplet PCR technology (Tewhey R. et al., Nature Biotech. 2009, 27:1025-1031), custom-designed oligonucleotide microarrays (e.g., Roche/NimbleOen oligonucleotide microarrays), and solution-based hybridization methods (e.g., molecular inversion probes (MIPs) (Porreca O. J. et al., Nature Methods, 2007, 4:931-936; Krishnakumar S. et al., Proc. Natl. Acad. Sci. USA, 2008, 105:9296-9310; Turner E. H. et al., Nature Methods, 2009, 6:315-316), and biotinylated RNA capture sequences (Onirke A. et al., Nat. Biotechnol. 2009; 27(2): 182-9).

In some embodiments, the method comprises a step of contacting the sample with one or more baits or bait sets to provide a selected library catch. The contacting step can be effected in solution hybridization. In certain embodiments, the method includes repeating the hybridization step by one or more additional rounds of solution hybridization. In some embodiments, the methods further include subjecting the library catch to one or more additional rounds of solution hybridization with the same or different collection of baits. Hybridization methods that can be adapted for use in the methods herein are described in the art, e.g., as described in International Patent Application Publication No. WO 2012/092426. In other embodiments, the method comprises amplifying the library catch (e.g., by PCR). In other embodiments, the library catch is not amplified. The library catch or a subgroup thereof can be sequenced. In certain embodiments, the library catch can be re-sequenced.

Exemplary sequencing and imaging methods for NGS include, but are not limited to, cyclic reversible termination (CRT), sequencing by ligation (SBL), single-molecule addition (pyrosequencing), and real-time sequencing. Other sequencing methods for NGS include, but are not limited to, nanopore sequencing, sequencing by hybridization, nano-transistor array based sequencing, polony sequencing, scanning tunneling microscopy (STM) based sequencing, and nanowire-molecule sensor based sequencing. Sequencing methods suitable for use herein are described in the art, e.g., as described in International Patent Application Publication No. WO 2012/092426.

After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild-type sequence). Methods of sequence alignment for NGS are described e.g., in Trapnell C. and Salzberg S.L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data. After alignment, detection of mutations such as substitutions can he performed using a calling method, e.g., Bayesian mutation calling method; which is applied to each base in each of the subgenomic intervals, e.g., exons of the gene to be evaluated, where presence of alternate alleles is observed. Algorithms and methods for data analysis, including sequence alignment and mutation calling, are described in WO2012/092426, incorporated herein by reference.

### C. Therapy

Certain aspects of the present disclosure relate to treatment of meningioma.

Current treatment options for meningioma depends on a variety of factors, including: (1) the size and location of meningioma; (2) rate of growth or aggressiveness of the tumor; and (3) age and overall health of the patient. Standard treatments include surgery, or surgery combined with radiation therapy. Tumor resection refers to physical removal of at least part of a tumor. In addition to tumor resection, treatment by surgery includes laser surgery, cryosurgery, electrosurgery, and microscopically-controlled surgery (Mohs' surgery). The completeness of tumor resection in a surgery is graded using routing Simpson grading (Simpson D. J. Neurol. Neurosurg. Psychiatry. 1957; 20: 22-39). Grade I tumor resection refers to macroscopically complete resection with excision of dural attachment and abnormal bone. Grade II tumor resection refers to macroscopically complete resection with coagulation of dural attachment. Grade III tumor resection refers to macroscopically complete resection without resection or coagulation of its attachment. Grade IV tumor resection refers to subtotal resection. Grade V tumor resection refers to simple decompression of the tumor, including biopsy. The level of surgical resection applied to a meningioma patient depends on factors such as aggressiveness of the tumor and location of the tumor. Evaluation of resection completeness can be made intraoperatively, and can be confirmed postoperatively through methods known in the art, such as by contrast-enhanced magnetic resonance imaging (MRI) or computer tomography (CT) scans.

In some embodiments, the method comprises subjecting the individual to aggressive tumor resection based on the presence of an inactivating mutation of PBRM1 in the individual, e.g., in the meningioma of the individual. In some embodiments, the individual is subject to Simpson Grade I tumor resection. In some embodiments, the individual is subject to Simpson Grade II tumor resection. In some embodiments, the individual is subject to Simpson Grade III tumor resection. In some embodiments, the individual would have been subject to a lower grade (i.e., less complete) tumor resection based on histological or imaging diagnosis of the meningioma.

In some embodiments, the method comprises administering to the individual an effective amount of an adjuvant therapy based on the presence of an inactivating mutation of PBRM1 in the individual, e.g., in the meningioma of the individual. Adjuvant therapy may have undesirable side effects, and may not be indicated for the individual based on histological or imaging diagnosis of the meningioma. Exemplary adjuvant therapies include, but are not limited to, targeted therapies, chemotherapies, anti-angiogenic agents, radiotherapies, anti-inflammatory therapies, cancer immunotherapies, and combinations thereof. In some embodiments, the adjuvant therapy comprises administering to the individual one or more agents selected from the group consisting of an anti-neoplastic agent, a chemotherapeutic agent, a growth inhibitory agent, an anti-angiogenic agent, a radiotherapy agent, and a cytotoxic agent. In some embodiments, the individual is administered an adjuvant therapy following surgery. In some embodiments, the individual is administered an adjuvant therapy following surgery and radiotherapy.

In some embodiments, the adjuvant therapy comprises radiotherapy (also referred herein as radiation therapy). In some embodiments, the radiotherapy is stereotactic radiosurgery (SRS). In some embodiments, the radiotherapy is fractionated stereotactic radiotherapy (SRT). In some embodiments, the radiotherapy is intensity-modulated radiation therapy (IMRT). In some embodiments, the radiotherapy is proton beam radiation.

The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, De Vita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In some embodiments, the adjuvant therapy comprises a targeted therapy. The term “targeted therapy” refers to administration of agents that selectively interact with a chosen biomolecule to thereby treat cancer. For example, anti-PBRM1 agents, such as therapeutic monoclonal blocking antibodies, which are well-known in the art and described above, can be used to target tumor microenvironments and cells expressing unwanted PBRM1.

In some embodiments, the adjuvant therapy comprises a chemotherapy. Chemotherapy includes the administration of a chemotherapeutic agent.

Exemplary chemotherapeutic agents include, but are not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In some embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamine (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP 1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In some embodiments, the adjuvant therapy comprises an anti-inflammatory therapy. In some embodiments, the anti-inflammatory agent is an agent that blocks, inhibits, or reduces inflammation or signaling from an inflammatory signaling pathway.

In some embodiments, an anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23, interferons (IFNs), e.g., IFNα, IFNβ, IFNγ, IFN-≢5 inducing factor (IGIF), transforming growth factor-β (TGF-β), transforming growth factor-α (TGF-α), tumor necrosis factors TNF-α, TNF-β, TNF-RI, TNF-RII, CD23, CD30, CD40L, EGF, G-CSF, GDNF, PDGF-BB, RANTES/CCL5, IKK, NF-κB, TLR2, TLR3, TLR4, TL5, TLR6, TLR7, TLR8, TLR8, TLR9, and/or any cognate receptors thereof. In some embodiments, the anti-inflammatory agent is an IL-1 or IL-1 receptor antagonist, such as anakinra (KINERET®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (REMICADE®), golimumab (SIMPONI®), adalimumab (HUMIRA®), certolizumab pegol (CIMZIA®) or etanercept.

In some embodiments, an anti-inflammatory agent is a corticosteroid. Exemplary corticosteroids include, but are not limited to, cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PREZONE®), and prednisone (DELTASONE®, LIQUID PRED®, METICORTEN®, ORASONE®)), and bisphosphonates (e.g., pamidronate (AREDIA), and zoledronic acid (ZOMETAC®).

In some embodiments, the method comprises administering to the individual an effective amount of an anti-cancer agent based on the presence of an inactivating mutation of PBRM1 in the individual. In some embodiments, the anti-cancer agent is selected from the group consisting of an anti-angiogenic agent, a microtubule-destabilizing agent, a chemotherapeutic agent, an anti-DNA repair agent, and an anti-inflammatory agent.

In some embodiments, the method comprises administering to the individual an effective amount of an anti-angiogenic agent based on the presence of an inactivating mutation of PBRM1 in the individual. In some embodiments, the anti-angiogenic agent anti-angiogenic agent is selected from the group consisting of axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept.

Anti-angiogenic agents prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or anti-angiogenic agents that can be used in the methods described herein include soluble VEGF (VEGF isoforms VEGF121 and VEGF165, receptors VEGFR1, VEGFR2 and co-receptors Neuropilin-1 and Neuropilin-2) 1 and NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, -β and -γ, CXCL10, IL-4, -12 and -18, prothrombin (kringle domain-2), antithrombin III fragment, prolactin, VEGI, SPARC, osteopontin, maspin, canstatin, proliferin-related protein, restin and drugs like e.g. bevacizumab, itraconazole, carboxyamidotriazole, TNP-470, CM101, suramin, SU5416, thrombospondin, VEGFR antagonists, angiostatic steroids+heparin, cartilage-derived angiogenesis Inhibitory factor, matrix metalloproteinase inhibitors, 2-methoxyestradiol, tecogalan, tetrathiomolybdate, thalidomide, thrombospondin, prolactina v β3 inhibitors, linomide, and tasquinimod. For review see Schoenfeld and Dranoff 2011: Anti-angiogenesis immunotherapy. Hum Vaccin. (9):976-81; Al-Husein et al., Anti-angiogenic therapy for cancer: An update; Pharmacotherapy 32(12): 1095-1111 (2012).

In some embodiments, the anti-angiogenic agent is a naturally occurring angiogenic inhibitor, such as, angiostatin, endostatin, or platelet factor-4. In some embodiments, the anti-angiogenic agent is a specific inhibitor of endothelial cell growth, such as TNP-470, thalidomide, or interleukin-12. Other suitable anti-angiogenic agents include those that neutralize angiogenic molecules, such as including without limitation, antibodies to fibroblast growth factor, antibodies to vascular endothelial growth factor, antibodies to platelet derived growth factor, and antibodies or other types of inhibitors of the receptors of EGF, VEGF or PDGF.

In some embodiments, the anti-angiogenic agent is suramin or an analog thereof, or tecogalan. In some embodiments, the anti-angiogenic agent is an agent that neutralizes a receptor for an angiogenic factor, or an agent that interferes with vascular basement membrane and extracellular matrix, such as a metalloprotease inhibitor, or an angiostatic steroid. Another group of anti-angiogenic agents includes, without limitation, anti-adhesion molecules, such as antibodies to integrin alpha v beta 3. Still other anti-angiogenic agents, include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In some embodiments, the anti-angiogenic agent is an antibody to VEGF, such as bevacizumab (AVASTIN®).

In some embodiments, the method comprises administering to the individual an effective amount of a microtubule-destabilizing agent based on the presence of an inactivating mutation of PBRM1 in the individual. In some embodiments, the microtubule-destabilizing agent is selected from the group consisting of vinblastine, vincristine, vinorelbine, vinflunine, cryptophycins (e.g., cryptophycin 52), halichondrins, dolastatins, hemiasterlins, colchicine, combretastatins, 2-methoxyestradiol, E7010, ombrabulin, soblidotin, D-24851, pseudolaric acid B, and embellistatin.

Microtubule-destabilizing agents are agents that depolymerize microtubules, e.g., by interacting with various β-tubulin sites. Two exemplary classes of microtubule-destabilizing agents are colchicine and vinca alkaloids. Vinca alkaloids interact with tubulin at specific binding sites which differ from those of other agents, including colchicine or taxanes, interfering with microtubule dynamics, blocking polymerization at the end of the mitotic spindle, and leading to metaphase arrest. Exemplary vinca alkaloids include, but are not limited to, vinblastine, vinorelbine, vincristine, and vindesine. Another group of microtubule-stabilizing agents are cryptophycins, which are synthetic derivatives of macrocyclic depsipeptides, isolated from Nostoc sp. They block cell division and prevent the correct formation of the mitotic spindle, by inhibiting tubulin polymerization, probably at the binding site of the Vinca alkaloids. Exemplary cryptophycins include, but are not limited to, C-52, C-55, C-309, C-249, and C-283. Combretastatins, isolated from Combretum caffrum, are another group of microtubule-destabilizing agents, which are structurally related to colchicine. Other microtubule-destabilizing agents include, but are not limited to, ombrabulin, dolastatins (e.g., dolastatin 10 and 15), soblidotin (TZT-1027), cemadotin (LU103793), tasidotin (ILX651), rhizoxin (NSC332598), indibulin (D-24851), pseudolaric acid B (PAB), embellistatin, CI-980, T138067, T138067, and ABT-751 (E7010). For review, see, Fanale D. et al., Stabilizing versus Destabilizing the Microtubules: A Double-Edge Sword for an Effective Cancer Treatment Option? Analytical Cellular Pathology, 2015: Article ID690916.

In some embodiments, the method comprises administering to the individual an effective amount of a cancer immunotherapy based on the presence of an inactivating mutation of PBRM1 in the individual, e.g., in the meningioma of the individual. In some embodiments, the cancer immunotherapy comprises one or more immunotherapies selected from the group consisting of a checkpoint inhibitor, cancer vaccine, cell-based therapy, T cell receptor (TCR)-based therapy, adjuvant immunotherapy, cytokine immunotherapy, and oncolytic virus therapy. In some embodiments, the cancer immunotherapy comprises small molecule, nucleic acid, polypeptide, carbohydrate, toxin, cell-based, or binding agent therapeutic agent. Examples of cancer immunotherapies are described in greater detail infra but are not intended to be limiting. In some embodiments, the cancer immunotherapy activates one or more aspects of the immune system to attack a cell (e.g., a tumor cell) that expresses a neoantigen of the present disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises cancer vaccine. In some embodiments, the cancer vaccine comprises a polynucleotide that encodes a neoantigen found in the meningioma of the individual. In some embodiments, the cancer vaccine is a peptide cancer vaccine, which in some instances is a personalized peptide vaccine. In some embodiments the peptide cancer vaccine is a multivalent long peptide, a multi-peptide, a peptide cocktail; a hybrid peptide, or a peptide-pulsed dendritic cell vaccine (see, e.g., Yamada et al., Cancer Sci. 104:14-21, 2013).

In some embodiments, the cancer immunotherapy comprises a cell-based therapy. In some embodiments, the cancer immunotherapy comprises a T cell-based therapy. In some embodiments, the cancer immunotherapy comprises an adoptive T cell-based therapy. In some embodiments, the T cells are autologous or allogeneic to the recipient. In some embodiments, the T cells are CD8+ T cells. In some embodiments, the T cells are CD4+ T cells.

In some embodiments, the T cell-based therapy comprises a chimeric antigen receptor (CAR)-T-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. TILs are then administered to the patient (optionally in combination with one or more cytokines or other immune-stimulating substances).

In some embodiments, the cell-based therapy comprises a dendritic cell-based therapy, e.g., a dendritic cell vaccine. Dendritic cell vaccines (such as Sipuleucel-T, also known as APC8015 and PROVENGE) are vaccines that involve administration of dendritic cells that act as APCs to present one or more cancer-specific antigens, e.g., a neoantigen of the present disclosure, to the patient's immune system. In some embodiments, the dendritic cells are autologous or allogeneic to the recipient.

In some embodiments, the cancer immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the cancer immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the cancer immunotherapy comprises oncolytic virus therapy. Oncolytic virus therapy uses genetically modified viruses to replicate in and kill cancer cells, leading to the release of antigens that stimulate an immune response.

In some embodiments, the cancer immunotherapy comprises a checkpoint inhibitor. As is known in the art, a checkpoint inhibitor targets at least one immune checkpoint protein to alter the regulation of an immune response, e.g., down-modulating or inhibiting an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIRL CD80, CD86, CD276, VTCN1, MHC class I, MHC class II, GALS, adenosine, TGFR, CSF1R, MICA/B, arginase, CD160, gp49B, PIR-B. KIR family receptors, TIM-1, TIM-3, TIM-4, LAG-3, BMA, SIRPalpha (CD47), CD48, 2B4 (CD244), 137.1, B7.2, ILT-2, ILT-4, BTLA, IDO, OX40, and A2aR. In some embodiments, a checkpoint inhibitor decreases the activity of a checkpoint protein that negatively regulates immune cell function, e.g., in order to enhance T cell activation and/or an anti-cancer immune response; in other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. In some embodiments, the checkpoint inhibitor is an antibody. Examples of checkpoint inhibitors include, without limitation, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof.

In some embodiments, the checkpoint inhibitor is a PD-L1 axis binding antagonist, e.g., a PD-1 binding antagonist, a PD-L1 binding antagonist, or a PD-L2. binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG-2,” “CD273,” “B7-DC,” “Btdc,” and “PDL2.” An exemplary human PD-L2 is shown in UniProtKB/Swiss-Prot Accession No. Q9BQ51. In some embodiments, PD-1, PD-L1, and PD-L2 are human PD-1, PD-L1 and PD-L2.

In some embodiments. the PD-1 binding antagonist is a molecule that inhibits the binding of PD-1 to its ligand binding partners. In a specific aspect the PD-1 ligand binding partners are PD-L1 and/or PD-L2. In another instance, a PD-L1 binding antagonist is a molecule that inhibits the binding of PD-L1 to its binding ligands. In a specific aspect, PD-L1 binding partners are PD-1 and/or B7-1. In another instance, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide. In some embodiments, the PD-1 binding antagonist is a small molecule, a nucleic acid, a polypeptide (e.g., antibody), carbohydrate, a lipid, a metal, or a toxin.

In some embodiments, the PD-1 binding antagonist is an anti-PD-1 antibody (e.g., a human antibody, a humanized antibody, or a chimeric antibody), for example, as described below. In some embodiments, the anti-PD-1 antibody is selected from the group consisting of MDX-1 106 (nivolumab), MK-3475 (pembrolizumab), MEDI-0680 (AMP-514), PDR001, REGN2810, MGA-012, JNJ-63723283, BI 754091, and BGB-108. MDX-1 106, also known as MDX-1 106-04, ONO-4538. BMS-936558, or nivolumab, is an anti-PD-1 antibody described in WO2006/121 168. MK-3475, also known as pembrolizumab or lambrolizumab, is an anti-PD-1 antibody described in WO 2009/1 14335. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. AMP-224, also known as B7-DCIg, is a PD-L2-Fc fusion soluble receptor described in WO 2010/027827 and WO 2011 /066342.

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335.

Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), ENUM 388D4 (Enumeral Biomedical Holdings). In some embodiments, the PD-1 binding antagonist is a peptide or small molecule compound. In some embodiments, the PD-1 binding antagonist is AUNP-12 (PierreFabre/Aurigene).

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In any of the instances herein, the isolated anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), carbohydrate, a lipid, a metal, or a toxin.

In some embodiments, the PD-L1 binding antagonist is an anti-PD-L1 antibody, for example, as described below. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, fv, scFv, and (Fab′)2 fragments. In some embodiments, the anti-PD-L1 antibody is a humanized antibody. In some embodiments, the anti-PD-L1 antibody is a human antibody. In some embodiments, the anti-PD-L1 antibody is selected from the group consisting of YW243.55.S70, MPDL3280A (atezolizumab), MDX-1 105, and MEDI4736 (durvalumab), and MSB0010718C (avelumab). Antibody YW243.55.S70 is an anti-PD-L1 described in WO 2010/077634. MDX-1 105, also known as BMS-936559, is an anti-PD-L1 antibody described in WO2007/005874. MEDI4736 (durvalumab) is an anti-PD-L1 monoclonal antibody described in WO201 1 /066389 and US2013/034559. Examples of anti-PD-L1 antibodies useful for the methods described herein, and methods for making thereof are described in PCT patent application WO 2010/077634, WO 2007/005874, WO 2011/066389, U.S. Pat. No. 8,217,149, and US2013/034559.

In some embodiments, the anti-PDL1 antibody is MPDL3280A, also known as atezolizumab and TECENTRIQ® (CAS Registry Number: 1422185-06-5).

In a still further specific aspect, the antibody further comprises a human or murine constant region. In a still further aspect, the human constant region is selected from the group consisting of lgG1, lgG2, lgG2, lgG3, and lgG4. In a still further specific aspect, the human constant region is lgG1. In a still further aspect, the murine constant region is selected from the group consisting of lgG1, lgG2A, lgG2B, and lgG3. In a still further aspect, the murine constant region in lgG2A. In a still further specific aspect, the antibody has reduced or minimal effector function. In a still further specific aspect the minimal effector function results from an “effector-less Fc mutation” or aglycosylation. In still a further instance, the effector-less Fc mutation is an N297A or D265A/N297A substitution in the constant region.

In some embodiments, the anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acrid residue e.g., glycine, alanine or a conservative substitution).

In some embodiments, the anti-PDL1 antibody is avelumab (CAS Registry Number: 1537032-82-8). Avelumab, also known as MSB0010718C, is a human monoclonal IgG1 anti- PDL1 antibody (Merck KGaA, Pfizer).

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559.

Other examples of anti-PD-L1 antibodies include, but are not limited to, MDX-1105 (BMS-936559; Bristol Myers Squibb), LY3300054 (Eli Lilly), STI-A1014 (Sorrento), KN035 (Suzhou Alphamab), FAZ053 (Novartis), or CX-072 (CytomX Therapeutics).

In some embodiments, the checkpoint inhibitor is CT-011, also known as hBAT, hBAT-1 or pidilizumab, an antibody described in WO 2009/101611.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), carbohydrate, a lipid, a metal, or a toxin.

In some embodiments, the anti-CTLA4 antibody is ipilimumab (YERVOY®; CAS Registry Number: 477202-00-9). Ipilimumab, also known as BMS-734016, MDX-010, and MDX-101, is a fully human monoclonal IgG1 kappa anti-CTLA4 antibody (Bristol-Myers Squibb) described in WO2001/14424.

Other examples of anti-CTLA4 antibodies include, but are not limited to, APL-509, AGEN1884, and CS1002.

In some embodiments, the individual has received a prior therapy for meningioma. The prior therapy may comprise one or more therapies selected from the group consisting of surgery, a targeted therapy, a chemotherapy, an anti-angiogenic agent, radiotherapy, an anti-inflammatory therapy, an anti-DNA repair therapy, and a cancer immunotherapy, as described above. The individual may have received any number of cycles of prior therapy, or combination of prior therapies.

Anti-DNA repair therapies include, but are not limited to, a PARP inhibitor, a RAD51 inhibitor, or an inhibitor of a DNA damage response kinase selected from CHCK1, ATM, or ATR. In some embodiments, the anti-DNA repair therapy is flash radiation therapy in combination with a radiosensitizer. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. SSB repair mechanisms include BER, NER, or MMR pathways whilst DSB repair mechanisms consist of HR and NHEJ pathways. Radiation causes DNA breaks that if not repaired are lethal. Single strand breaks are repaired through a combination of BER, NER and MMR mechanisms using the intact DNA strand as a template. The predominant pathway of SSB repair is the BER utilizing a family of related enzymes termed poly-(ADP-ribose) polymerases (PARP). In some embodiments, the anti-DNA repair therapy is flash radiation therapy combined with a PARP inhibitor (e.g., Talazoparib, Rucaparib, Olaparib) (Lord and Ashworth, 2016; Murai et al., 2012), a RAD51 inhibitor (RI-1), or an inhibitor of DNA damage response kinases such as CHCK1 (AZD7762), ATM (KU-55933, KU-60019, NU7026, VE-821), and ATR (NU7026).

The therapeutic agents and compositions thereof utilized in the methods described herein e.g., adjuvant therapies, anti-angiogenic agents, microtubule-destabilizing agents, or cancer immunotherapies) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratutnorally, peritoneally, subconjunctival, intravesicularly, mucosally, intrapericardially, intraumbilically, intraocularly, intraorbitally, orally, topically, transdermal, intravitreally, by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The adjuvant therapies, anti-angiogenic agents, microtubule-destabilizing agents, and cancer immunotherapies (e.g., an antibody, binding polypeptide, and/or small molecule) described herein may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the therapeutic agent present in the formulation, the type of disorder or treatment, and other factors discussed above. For example, as a general proposition, the therapeutically effective amount of an immune checkpoint inhibitor, for example, a PD-L1 axis binding antagonist antibody, an anti-CTLA-4 antibody, an anti-TIM-3 antibody, or an anti-LAG-3 antibody, administered to human will be in the range of about 0.01 to about 50 mg/kg of patient body weight, whether by one or more administrations. In some embodiments, anti-PD-L1 antibody MPDL3280A is administered at 1200 mg intravenously in three week (q3w) intervals. The dose may be administered as a single dose or as multiple doses (e.g., 2 or 3 doses), such as infusions. The dose of therapeutic agents administered in a combination treatment may be reduced as compared to a single treatment. The progress of this therapy is easily monitored by conventional techniques.

In some embodiments, the individual is subject to two or more therapies. In some embodiments, a cancer immunotherapy may be administered in conjunction with a chemotherapy or chemotherapeutic agent. In some embodiments, a cancer immunotherapy may be administered in conjunction with a radiation therapy agent. In some embodiments, a cancer immunotherapy may be administered in conjunction with a targeted therapy or targeted therapeutic agent. In some embodiments, a cancer immunotherapy may be administered in conjunction with another immunotherapy or immunotherapeutic agent, for example, an anti-PD1 antibody and an anti-CTLA4 antibody. In some embodiments, a cancer immunotherapy is administered with an agonist directed against a co-stimulatory molecule. In some embodiments, a cancer immunotherapy is administered with an antagonist directed against a co-inhibitory molecule. In some embodiments, the individual is administered a monotherapy, for example, the cancer immunotherapy is administered as a monotherapy.

Such combination therapies noted above encompass combined administration (where two or more therapeutic agents are included in the same or separate formulations), and separate administration, in which case, administration of a cancer immunotherapy can occur prior to, simultaneously, and/or following, administration of a radiation therapeutic agent. In some embodiments, administration of different therapeutic agents occur within about one month, or within about one, two or three weeks, or within about one, two, three, four, five, or six days, of each other.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some embodiments, an immune checkpoint inhibitor, for example, a PD-L1 axis binding antagonist and/or CTLA4 antagonist, may be administered in conjunction with an agonist directed against a co-stimulatory molecule. In some embodiments, a co-stimulatory molecule may include CD40, CD226, CD28, OX40, GITR, CD137, CD27, HVEM, or CD127. In some embodiments, the agonist directed against a co-stimulatory molecule is an agonist antibody that binds to CD40, CD226, CD28, OX40, GITR, CD137, CD27, HVEM, or CD127. In some embodiments, an immune checkpoint inhibitor, for example, a PD-L1 axis binding antagonist and/or CTLA4 antagonist, may be administered in conjunction with an antagonist directed against a co-inhibitory molecule. In some embodiments, a co-inhibitory molecule may include CTLA-4 (also known as CD1 52), TIM-3, BTLA, VISTA, LAG-3, B7-H3, B7-H4, IDO, TIGIT, MICA/B, or arginase. In some embodiments, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some embodiments, a PD-L1 axis binding antagonist may be administered in conjunction with an antagonist directed against CTLA-4 (also known as CD152), e.g., a blocking antibody. In some embodiments, a PD-L1 axis binding antagonist may be administered in conjunction with ipilimumab (also known as MIDX-010, MDX-101, or YERVOY®). In some embodiments, a PD-L1 axis binding antagonist may be administered in conjunction with tremelimumab (also known as ticilimumab or CP-675,206). In some embodiments, a PD-L1 axis binding antagonist may be administered in conjunction with an antagonist directed against B7-H3 (also known as CD276), e.g., a blocking antibody. In some embodiments, a PD-L1 axis binding antagonist may be administered in conjunction with MGA271. In some embodiments, a PD-L1 axis binding antagonist may be administered in conjunction with an antagonist directed against a TGF-beta, metelimumab (also known as CAT-192), fresolimumab (also known as GC1008), or LY2157299.

In some embodiments, an immune checkpoint inhibitor, for example, a PD-L1 axis binding antagonist and/or CTLA4 antagonist, may be administered in conjunction with a treatment comprising adoptive transfer of a T-cell (e.g., a cytotoxic T-cell or CTL) expressing a chimeric antigen receptor (CAR). In some embodiments, an immune checkpoint inhibitor, for example, a PD-L1 axis binding antagonist and/or CTLA4 antagonist, may be administered in conjunction with a treatment comprising adoptive transfer of a T-cell comprising a dominant-negative TGF beta receptor, e.g., a dominant-negative TGF beta type II receptor. In sonic embodiments, an immune checkpoint inhibitor, for example, a PD-L1 axis binding antagonist and/or CTLA4 antagonist, may be administered in conjunction with a treatment comprising a HERCREEM protocol (see, e.g., ClinicalTrials.gov Identifier NCT00889954).

In some embodiments, an immune checkpoint inhibitor, for example, a PD-L1 axis binding antagonist and/or CTLA4 antagonist, may be administered in conjunction with an agonist directed against CD137 (also known as TNFRSF9, 4-1 BB, or ILA), e.g., an activating antibody. In some embodiments, an immune checkpoint inhibitor, for example, a PD-L1 axis binding antagonist and/or CTLA4 antagonist, may be administered in conjunction with urelumab (also known as HMS-663513). In some embodiments, an immune checkpoint inhibitor, for example, a PD-L1 axis binding antagonist and/or CTLA4 antagonist, may be administered in conjunction with an agonist directed against CD40, e.g., an activating antibody. In some embodiments, an immune checkpoint inhibitor, for example, a PD-L1 axis binding antagonist and/or CTLA4 antagonist, may be administered in conjunction with CP-870893. In some embodiments, an immune checkpoint inhibitor, for example, a PD-L1 axis binding antagonist and/or CTLA4 antagonist, may be administered in conjunction with an agonist directed against OX40 (also known as CD134), e.g., an activating antibody. In some embodiments, an immune checkpoint inhibitor, for example, a PD-L1 axis binding antagonist and/or CTLA4 antagonist, may be administered in conjunction with an anti-OX40 antibody (e.g., AgonOX). In some embodiments, an immune checkpoint inhibitor, for example, a PD-L1 axis binding antagonist and/or CTLA4 antagonist, may be administered in conjunction with an agonist directed against CD27, e.g., an activating antibody. In some embodiments, an immune checkpoint inhibitor, for example, a PD-L1 axis binding antagonist and/or CTLA1 antagonist, may be administered in conjunction with CDX-1127. In some embodiments, an immune checkpoint inhibitor, for example, a PD-L1 axis binding antagonist and/or CTLA4 antagonist, may be administered in conjunction with an antagonist directed against indoleamine-2,3-dioxygenase (IDO). In some embodiments, with the IDO antagonist is 1-methyl-D-tryptophan (also known as 1-D-MT).

In some embodiments, an immune checkpoint inhibitor, for example, a. PD-L1 axis binding antagonist and/or CTLA4 antagonist, may be administered in conjunction with an antibody-drug conjugate.

In some embodiments, an immune checkpoint inhibitor, for example, a PD-L1 axis binding antagonist and/or CTLA4 antagonist, may be administered in conjunction with an anti-angiogenesis agent. In some embodiments, an immune checkpoint inhibitor, for example, a PD-L1 axis binding antagonist and/or CTLA4 antagonist, may be administered in conjunction with an antibody directed against a VEGF, e.g., VEGF-A. In some embodiments, an immune checkpoint inhibitor, for example, a PD-L1 axis binding antagonist and/or CTLA4 antagonist, may be administered in conjunction with bevacizumab (also known as AVASTIN®, Genentech). In some embodiments, an immune checkpoint inhibitor, for example, a PD-L1 axis binding antagonist and/or CTLA4 antagonist, may be administered in conjunction with an antibody directed against angiopoietin 2 (also known as Ang2). In some embodiments, an immune checkpoint inhibitor, for example, a PD-L1 axis binding antagonist and/or CTLA4 antagonist, may be administered in conjunction with MEDI3617.

In some embodiments, an immune checkpoint inhibitor, for example, a PD-L1 axis binding antagonist ane/or CTLA4 antagonist, may be administered in conjunction with an antineoplastic agent. In some embodiments, an immune checkpoint inhibitor, for example, a PD-L1 axis binding antagonist and/or CTLA4 antagonist, may be administered in conjunction with an agent targeting CSF-1R (also known as M-CSFR or CD1 15). In some embodiments, an immune checkpoint inhibitor, for example, a PD-L1 axis binding antagonist and/or CTLA4 antagonist, may be administered in conjunction with anti-CSF-1 R (also known as IMC-CS4). In some embodiments, an immune checkpoint inhibitor, for example, a PD-L1 axis binding antagonist and/or CTLA4 antagonist, may be administered in conjunction with an interferon, for example interferon alpha or interferon gamma. In some embodiments, an immune checkpoint inhibitor, for example, a PD-L1 axis binding antagonist and/or CTLA4 antagonist, may be administered in conjunction with Roferon-A (also known as recombinant Interferon alpha-2a). In some embodiments, an immune checkpoint inhibitor, for example, a PD-L1 axis binding antagonist and/or CTLA4 antagonist, may be administered in conjunction with GM-CSF (also known as recombinant human granulocyte macrophage colony stimulating factor, rhu GM-CSF, sargramostim, or LEUKINE®). In some embodiments, an immune checkpoint inhibitor, for example, a PD-L1 axis binding antagonist and/or CTLA4 antagonist, may be administered in conjunction with IL-2 (also known as aldesleukin or PROLEUKIN®). In some embodiments, an immune checkpoint inhibitor, for example, a PD-L1 axis binding antagonist and/or CTLA4 antagonist, may be administered in conjunction with IL-12. In some embodiments, an immune checkpoint inhibitor, for example, a PD-L1 axis binding antagonist and/or CTLA4 antagonist, may be administered in conjunction with an antibody targeting CD20. In some embodiments, the antibody targeting CD20 is obinutuzutnab (also known as GA101 or GAZYVA®) or rituximab. In some embodiments, an immune checkpoint inhibitor, for example, a PD-L1 axis binding antagonist and/or CTLA4 antagonist, may be administered in conjunction with an antibody targeting GITR. In some embodiments, the antibody targeting GITR is TRX518.

In some embodiments, an immune checkpoint inhibitor, for example, a PD-L1 axis binding antagonist and/or CTLA4 antagonist, may be administered in conjunction with a cancer vaccine.

In some embodiments, an immune checkpoint inhibitor, for example, a PD-L1 axis binding antagonist and/or CTLA4 antagonist, may be administered in conjunction with an adjuvant. In some embodiments, an immune checkpoint inhibitor, for example, a PD-L1 axis binding antagonist and/or CTLA4 antagonist, may be administered in conjunction with a treatment comprising a TLR agonist, e.g., Poly-ICLC (also known as HILTONOL®), LPS, MPL, or CpG ODN. In some embodiments, an immune checkpoint inhibitor, for example, a PD-L1 axis binding antagonist and/or CTLA4 antagonist, may be administered in conjunction with tumor necrosis factor (TNF) alpha. In some embodiments, an immune checkpoint inhibitor, for example, a PD-L1 axis binding antagonist and/or CTLA4 antagonist, may be administered in conjunction with IL-1. In some embodiments, an immune checkpoint inhibitor, for example, a PD-L1 axis binding antagonist and/or CTLA4 antagonist, may be administered in conjunction with HMGB1. In some embodiments, an immune checkpoint inhibitor, for example, a PD-L1 axis binding antagonist and/or CTLA4 antagonist, may be administered in conjunction with an IL-10 antagonist. In some embodiments, an immune checkpoint inhibitor, for example, a PD-L1 axis binding antagonist and/or CTLA4 antagonist, may be administered in conjunction with an IL-4 antagonist. In some embodiments, an immune checkpoint inhibitor, for example, a PD-L1 axis binding antagonist and/or CTLA4 antagonist, may be administered in conjunction with an IL-13 antagonist. In some embodiments, an immune checkpoint inhibitor, for example, a PD-L1 axis binding antagonist and/or CTLA4 antagonist, may be administered in conjunction with an HVEM antagonist. In some embodiments, an immune checkpoint inhibitor, for example, a PD-L1 axis binding antagonist and/or CTLA4 antagonist, may be administered in conjunction with an ICOS agonist, e.g., by administration of ICOS-L, or an agonistic antibody directed against ICOS. In some embodiments, an immune checkpoint inhibitor, for example, a PD-L1 axis binding antagonist and/or CTLA4 antagonist, may be administered in conjunction with a treatment targeting CX3CL1. In some embodiments, an immune checkpoint inhibitor, for example, a PD-L1 axis binding antagonist and/or CTLA4 antagonist, may be administered in conjunction with a treatment targeting CXCL9. In some embodiments, an immune checkpoint inhibitor, for example, a PD-L1 axis binding antagonist and/or CTLA4 antagonist, may be administered in conjunction with a treatment targeting CXCL10. In some embodiments, an immune checkpoint inhibitor, for example, a PD-L1 axis binding antagonist and/or CTLA4 antagonist, may be administered in conjunction with a treatment targeting CCL5. In some embodiments, an immune checkpoint inhibitor, for example, a PD-L1 axis binding antagonist and/or CTLA4 antagonist, may be administered in conjunction with an LFA-1 or ICAM1 agonist. In some embodiments, an immune checkpoint inhibitor, for example, a PD-L1 axis binding antagonist and/or CTLA4 antagonist, may be administered in conjunction with a Selectin agonist.

In some embodiments, an immune checkpoint inhibitor, for example, a PD-L1 axis binding antagonist and/or CTLA4 antagonist, may be administered in conjunction with a targeted therapy. In some embodiments, an immune checkpoint inhibitor, for example, a PD-L1 axis binding antagonist and/or CTLA4 antagonist, may be administered in conjunction with an inhibitor of B-Raf. In some embodiments, an immune checkpoint inhibitor, for example, a PD-L1 axis binding antagonist and/or CTLA4 antagonist, may be administered in conjunction with vemurafenib (also known as ZELBORAF®). In some embodiments, an immune checkpoint inhibitor, for example, a PD-L1 axis binding antagonist and/or CTLA4 antagonist, may be administered in conjunction with dabrafenib (also known as TAFINLAR®). In some embodiments, an immune checkpoint inhibitor, for example, a PD-L1 axis binding antagonist and/or CTLA4 antagonist, may be administered in conjunction with erlotinib (also known as TARCEVA®). In some embodiments, an immune checkpoint inhibitor, for example, a PD-L1 axis binding antagonist and/or CTLA4 antagonist, may be administered in conjunction with an inhibitor of a MEK, such as MEK1 (also known as MAP2K1) or MEK2 (also known as MAP2K2). In some embodiments, an immune checkpoint inhibitor, for example, a PD-L1 axis binding antagonist and/or CTLA4 antagonist, may be administered in conjunction with cobimetinib (also known as GDC-0973 or XL-518). In some embodiments, an immune checkpoint inhibitor, for example, a PD-L1 axis binding antagonist and/or CTLA4 antagonist, may be administered in conjunction with tratnetinib (also known as MEKINIST®). In some embodiments, an immune checkpoint inhibitor, for example, a PD-L1 axis binding antagonist and/or CTLA4 antagonist, may be administered in conjunction with an inhibitor of K-Ras. In some embodiments, an immune checkpoint inhibitor, for example, a PD-L1 axis binding antagonist and/or CTLA4 antagonist, may be administered in conjunction with an inhibitor of c-Met. In some embodiments, an immune checkpoint inhibitor, for example, a PD-L1 axis binding antagonist and/or CTLA4 antagonist, may be administered in conjunction with onartuzumab (also known as MetMAb). In some embodiments, an immune checkpoint inhibitor, for example, a PD-L1 axis binding antagonist and/or CTLA4 antagonist, may be administered in conjunction with an inhibitor of Alk. In some embodiments, an immune checkpoint inhibitor, for example, a PD-L1 axis binding antagonist and/or CTLA4 antagonist, may be administered in conjunction with AF802 (also known as CH5424802 or alectinib). In some embodiments, an immune checkpoint inhibitor, for example, a PD-L1 axis binding antagonist and/or CTLA4 antagonist, may be administered in conjunction with an inhibitor of a phosphatidylinositol 3-kinase (PI3K). In some embodiments, an immune checkpoint inhibitor, for example, a PD-L1 axis binding antagonist and/or CTLA4 antagonist, may be administered in conjunction with BKM120.

In some embodiments, an immune checkpoint inhibitor, for example, a PD-L1 axis binding antagonist and/or CTLA4 antagonist, may be administered in conjunction with idelalisib (also known as GS-1101 or CAL-101). In some embodiments, an immune checkpoint inhibitor, for example, a PD-L1 axis binding antagonist and/or CTLA4 antagonist, may be administered in conjunction with perifosine (also known as KRX-0401). In some embodiments, an immune checkpoint inhibitor, for example, a PD-L1 axis binding antagonist and/or CTLA4 antagonist, may be administered in conjunction with an inhibitor of an Akt. In some embodiments, an immune checkpoint inhibitor, for example, a PD-L1 axis binding antagonist and/or CTLA4 antagonist, may be administered in conjunction with MK2206. In some embodiments, an immune checkpoint inhibitor, for example, a PD-L1 axis binding antagonist and/or CTLA4 antagonist, may be administered in conjunction with GSK690693. In some embodiments, an immune checkpoint inhibitor, for example, a PD-L1 axis binding antagonist and/or CTLA4 antagonist, may be administered in conjunction with GDC-0941. In some embodiments, an immune checkpoint inhibitor, for example, a PD-L1 axis binding antagonist and/or CTLA4 antagonist, may be administered in conjunction with an inhibitor of mTOR. In some embodiments, an immune checkpoint inhibitor, for example, a PD-L1 axis binding antagonist and/or CTLA4 antagonist, may be administered in conjunction with sirolimus (also known as rapamycin). In some embodiments, an immune checkpoint inhibitor, for example, a PD-L1 axis binding antagonist and/or CTLA4 antagonist, may be administered in conjunction with temsirolimus (also known as CCI-779 or TORISEL®). In some embodiments, an immune checkpoint inhibitor, for example, a PD-L1 axis binding antagonist and/or CTLA4 antagonist, may be administered in conjunction with everolimus (also known as RAD001). In some embodiments, an immune checkpoint inhibitor, for example, a PD-L1 axis binding antagonist and/or CTLA4 antagonist, may be administered in conjunction with ridaforolimus (also known as AP-23573, MK-8669, or deforolimus). In some embodiments, an immune checkpoint inhibitor, for example, a PD-L1 axis binding antagonist and/or CTLA4 antagonist, may be administered in conjunction with OSI-027. In some embodiments, an immune checkpoint inhibitor, for example, a PD-L1 axis binding antagonist and/or CTLA4 antagonist, may be administered in conjunction with AZD8055. In some embodiments, an immune checkpoint inhibitor, for example, a PD-L1 axis binding antagonist and/or CTLA4 antagonist, may be administered in conjunction with INK128. In some embodiments, an immune checkpoint inhibitor, for example, a PD-L1 axis binding antagonist and/or CTLA4 antagonist, may be administered in conjunction with a dual PI3K/mTOR inhibitor. In some embodiments, an immune checkpoint inhibitor, for example, a PD-L1 axis binding antagonist and/or CTLA4 antagonist, may be administered in conjunction with XL765. In some embodiments, an immune checkpoint inhibitor, for example, a PD-L1 axis binding antagonist and/or CTLA4 antagonist, may be administered in conjunction with GDC-0980. In some embodiments, an immune checkpoint inhibitor, for example, a PD-L1 axis binding antagonist and/or CTLA4 antagonist, may be administered in conjunction with BEZ235 (also known as NVP-BEZ235). In some embodiments, an immune checkpoint inhibitor, for example, a PD-L1 axis binding antagonist and/or CTLA4 antagonist, may be administered in conjunction with BGT226. In some embodiments, an immune checkpoint inhibitor, for example, a PD-L1 axis binding antagonist and/or CTLA4 antagonist, may be administered in conjunction with GSK2126458. In some embodiments, an immune checkpoint inhibitor, for example, a PD-L1 axis binding antagonist and/or CTLA4 antagonist, may be administered in conjunction with PF-04691502. In some embodiments, an immune checkpoint inhibitor, for example, a PD-L1 axis binding antagonist and/or CTLA4 antagonist, may be administered in conjunction with PE-05212384 (also known as PKI-587).

While PD-L1 axis binding antagonists and CTLA4 antagonists are called out supra as exemplary cancer immunotherapies, this is not intended to be limiting; any cancer immunotherapies of the present disclosure may be administered in conjunction with any of the other treatments described herein, or otherwise known in the art (subject to medical judgement).

### D. Diagnosis and Prognosis

Certain aspects of the present application relates to diagnostic assays, prognostic assays, and monitoring treatment or clinical trials of an individual having meningioma.

In some embodiments, provided herein is a diagnostic assay for determining an inactivating mutation of PBRM1 in a sample (e.g., blood, serum, tumor, CSF, or tissue) of an individual having meningioma, thereby determining whether the individual is likely to respond to a therapy selected from the group consisting of aggressive tumor resection, an adjuvant therapy (e.g., radiotherapy), an anti-cancer agent (e.g., an anti-angiogenic agent, or a microtubule-destabilizing agent), a cancer immunotherapy, and combinations thereof. Such assays can be used for prognostic or predictive purpose to thereby prophylactically treat an individual prior to the onset or after recurrence of meningioma, such as papillary meningioma, associated with an inactivating mutation of PBRM1. In some embodiments, the method comprises monitoring the influence of a therapy on the expression or activity of PBRM1.

In some embodiments, the methods described herein are useful for classifying a sample (e.g., from a subject) as associated with or at risk for responding to or not responding to a therapy described herein using a statistical algorithm and/or empirical data. In some embodiments, the statistical algorithm is a single learning statistical classifier system. For example, a single learning statistical classifier system can be used to classify a sample as a based upon a prediction or probability value and the presence or level of an inactivating mutation of PBRM1. Other suitable statistical algorithms are well known to those of skill in the art. For example, learning statistical classifier systems include a machine learning algorithmic technique capable of adapting to complex data sets (e.g., panel of markers of interest) and making decisions based upon such data sets. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptions such as multi-layer perceptions, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method further comprises sending the sample classification results to a clinician, e.g., an oncologist. In some embodiments, the diagnosis of a subject is followed by administering to the individual a therapeutically effective amount of a therapeutic agent based upon the diagnosis.

In some embodiments, the methods described herein are used to identify subjects having or at risk of developing malignant meningioma, such as papillary meningioma. The prognostic assays described herein can be used to identify a subject having or at risk for developing malignant meningioma based on the presence or absence of an inactivating mutation of PBRM1. Furthermore, the prognostic assays described herein can be used to determine whether a subject can be subjected to a therapy (e.g., standard therapy, aggressive tumor resection, an anti-cancer agent (e.g., an anti-angiogenic agent, or a microtubule-destabilizing agent), adjuvant therapy such as radiotherapy, cancer immunotherapy, or clinical trial) to treat meningioma based on the presence or absence of an inactivating mutation of PBRM1.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive). Responses may be assessed, for example for efficacy or in a neoadjuvant or adjuvant situation, where the size of a tumor after systemic intervention can be compared to the initial size and dimensions as measured by CT, PET, or MRI. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR±PR+SD over 6 months. In some embodiments, the CBR for a particular cancer therapeutic regimen is at least 25%, 30%,35%, 40%, 45%, 50%, 55%, 60%, 65%, 70%, 75%, 80%>, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence includes both localized and distant recurrence); metastasis free survival; disease free survival (wherein the term disease includes cancer and diseases associated therewith). The length of said survival may be calculated by reference to a defined start point (e.g., time of diagnosis or start of treatment) and end point (e.g,, death, recurrence or metastasis). In addition, criteria for efficacy of treatment can be expanded to include response to chemotherapy, probability of survival, probability of metastasis within a given time period, and probability of tumor recurrence. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### E. Systems or Devices

The present application also provides a system or a device (e.g., a sequencing device) for producing a report, e.g., a report for recording the presence or absence of an inactivating mutation in PBRM1 in a patient. In some embodiments, the present application provides a system or a device (e.g., a sequencing device) for producing a report, e.g., a genotype report. The system or device can include a component for containing a sample (e.g., a blood or serum sample, or tumor sample from a patient); a detection component capable of identifying the presence or absence of an inactivating mutation in PBRM1; and a means for outputting a report, e.g., a report as described herein. For example, the detection component can include a probe or primer for detecting an inactivating mutation in PBRM1, such as by a sequencing method or by PCR. In some embodiments, the detection component is capable of identifying the presence or absence of mutations in one or more genes other than PBRM1.

In some embodiments, the component for containing a tumor sample is configured in a way to contain or hold the sample, e.g., a tumor nucleic acid or polypeptide sample.

In some embodiments, the detection component produces and/or analyzes a signal according to the presence or absence of an inactivating mutation in PBRM1 in the sample. In some embodiments, the detection component produces and/or analyzes a signal according to the presence or absence of mutations in one or more genes other than PBRM1.

In some embodiments, the means for outputting a report provides a system for annotating the association of the detected inactivating mutation in PBRM1 and optionally mutations in one or more genes other than PBRM1 to the sample. The report can include, e.g., the identification of nucleotide values, and the indication of presence or absence of a mutant gene as described herein, or sequence. In some embodiments, a report is generated, such as in paper or electronic form, which identifies the presence or absence of an alteration described herein, and optionally includes an identifier for the patient from which the sequence was obtained.

The report can also include information on the role of a sequence, e.g., an inactivating mutation in PBRM1 as described herein, or wild-type sequence, in cancer (e.g., meningioma). Such information can include information on prognosis, resistance, or potential or suggested therapeutic options, including clinical trials. The report can include information on the likely effectiveness of a therapeutic option, the acceptability of a therapeutic option, or the advisability of applying the therapeutic option to the patient.

### IV. Kits and Articles of Manufacture

Provided herein are kits including one or more reagents for detecting an inactivating mutation of PBRM1, as described herein, in a sample from an individual having meningioma. In some embodiments, the reagents include one or more oligonucleotides, e.g., for hybridization with DNA, RNA, or cDNA encoding any of the inactivating mutations of PBRM1 of the present disclosure. In some embodiments, the one or more reagents are nucleic acid molecules, baits, probes or primers as described herein. In some embodiments, the inactivating mutation of PBRM1 is p.F732fs*13, p.R146*, p.A482fs*18, p.Q949fs*59, p.E1029fs*100, p.K1372*, p.S39fs*14, p.S652fs*13, p.L1565fs*31, or p.V964fs*18. In some embodiments, the one or more reagents are antibodies that specifically binds to a PBRM1 protein.

Optionally, the kit may further include instructions to use the kit to select a therapy (e.g., aggressive tumor resection, adjuvant therapy such as radiotherapy, anti-angiogenic agent, microtubule-destabilizing agent, or cancer immunotherapy such as immune checkpoint inhibitor), or any combination thereof, for treating meningioma if the presence of an inactivating mutation of PBRM1 is detected.

Provided herein are also articles of manufacture including, packaged together, an adjuvant therapy (e.g., radiotherapy), an anti-cancer agent (e.g., an anti-angiogenic agent, or a microtubule-destabilizing agent), or a cancer immunotherapy (e.g., immune checkpoint inhibitor) in a pharmaceutically acceptable carrier and a package insert indicating that the adjuvant therapy, the anti-angiogenic agent, the microtubule-destabilizing agent, or the cancer immunotherapy is for treating an individual having meningioma based at least in part on the presence of an inactivating mutation of PBRM1 detected in a sample from the individual. Treatment methods include any of the treatment methods disclosed herein. The present application also provides a method for manufacturing an article of manufacture comprising combining in a package a pharmaceutical composition comprising an adjuvant therapy (e.g., radiotherapy), an anti-cancer agent (e.g., an anti-angiogenic agent, or a microtubule-destabilizing agent), or a cancer immunotherapy (e.g., immune checkpoint inhibitor), and a package insert indicating that the pharmaceutical composition is for treating an individual having meningioma based on the presence of an inactivating mutation of PBRM1 detected in a sample from the individual.

The article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising the cancer medicament as the active agent and may have a sterile access port (e.g., the container may be an intravenous solution bag or a vial having a stopper pierceable by a hypodermic injection needle).

The article of manufacture may further include a second container comprising a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The article of manufacture of the present application may also include information, for example in the form of a package insert, indicating that the composition is used for treating meningioma, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Frequent Inactivating Mutations of the PBAF Complex Gene PBRM1 in Meningioma with Papillary Features

Papillary meningioma (PM) is a World Health Organization (WHO) grade III meningioma subtype defined histologically by a predominant perivascular pseudopapillary growth pattern (Louis, D. N. et al. Acta Neuropathol. 2016 131:6). A papillary growth pattern in meningiomas has been associated with brain invasion and aggressive clinical behavior (Pasquier, B. et al. Cancer 1986 58:2; Kros, J. M. et al. Acta Neurol Scand. 2000 102:3; Avninder, S. et al. Diagn Pathol. 2007 2:3; Hojo, H. & Abe, M. Am J Surg Pathol. 2001 25:7). Another WHO grade III meningioma is the rhabdoid subtype which often harbors mutations in BAP1 (Shankar, G. M. et al. Neuro Oncol. 2017 19:4; Shankar, G. M. & Santagata, S. Neuro Oncol. 2017 19:11). Interestingly, some meningiomas have cells with rhabdoid cytomorphology arranged in a papillary architecture suggesting a potential molecular and genetic link or overlap between the papillary and rhabdoid histologic subtypes of meningioma (Hojo, H. & Abe, M. Am J Surg Pathol. 2001 25:7; Shankar, G. M. et al. Neuro Oncol. 2017 19:4).

The following example describes the identification of the PBRM1 gene as recurrently altered in meningiomas with papillary histomorphology.

### Materials and Methods

8 PM (>50% papillary morphology) and 22 meningiomas with focal papillary features (10-50%) amongst over 500 additional meningiomas of other subtypes were analyzed by clinical comprehensive genomic profiling (CGP).

Samples were analyzed in a CAP/CLIA-accredited laboratory (Foundation Medicine, Cambridge, MA). Approval for this study, including a waiver of informed consent and a HIPAA waiver of authorization, was obtained from the Western Institutional Review Board (Protocol No. 20152817). Three board-certified neuropathologists confirmed the pathologic diagnosis of each case on routine hematoxylin and eosin-stained slides. DNA was extracted from 40-μm-thick paraffin-embedded sections, and CGP was performed on hybridization-captured, adaptor ligation-based libraries to a mean coverage depth of >650× for 236 or 315 genes plus the introns from 19 or 28 genes respectively which are frequently involved in cancer (Trabucco, S. E. et al. J. Mol. Diagn. 2019 21:6). Tumor mutational burden (TMB) was determined on up to 1.14 megabases (Mb) of sequenced DNA by using an estimation algorithm that extrapolates to the genome as a whole (Frampton, G. M. et al. Nat Biotechnol. 2013 31:11; He, J. et al. Blood 2016 127:24; Forbes, S. A. et al. Nucleic Acids Res. 2011 39). Microsatellite instability (MSI) was determined on 114 loci (Forbes, S. A. et al. Nucleic Acids Res. 2011 39).

### Results

In the group of 8 PMs, three cases were identified with inactivation of PBRM1; two cases were identified with a truncating mutation in PBRM1 and one case was identified with homozygous deletion of PBRM1. Of the 22 meningiomas with only focal papillary features, 8 cases were PBRM1-mutants. Thus, 11 of 30 cases with at least focal (>10%) papillary morphology had inactivation of PBRM1.

In the entire cohort of 562 meningiomas, five additional cases with inactivating alterations in PBRM1 that did not display overt papillary morphology in the hematoxylin and eosin-stained sections available for analysis were identified. Thus, 11 of 16 PBRM1-mutant cases (69%) occurred in meningioma with papillary histologic features, which supported a significant association between papillary features and PBRM1 mutation (p<0.0001).

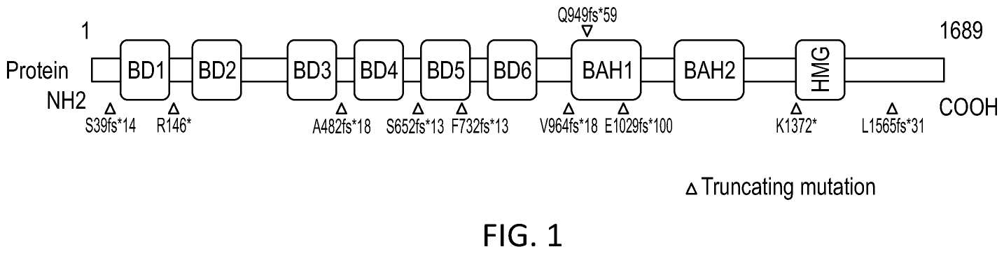

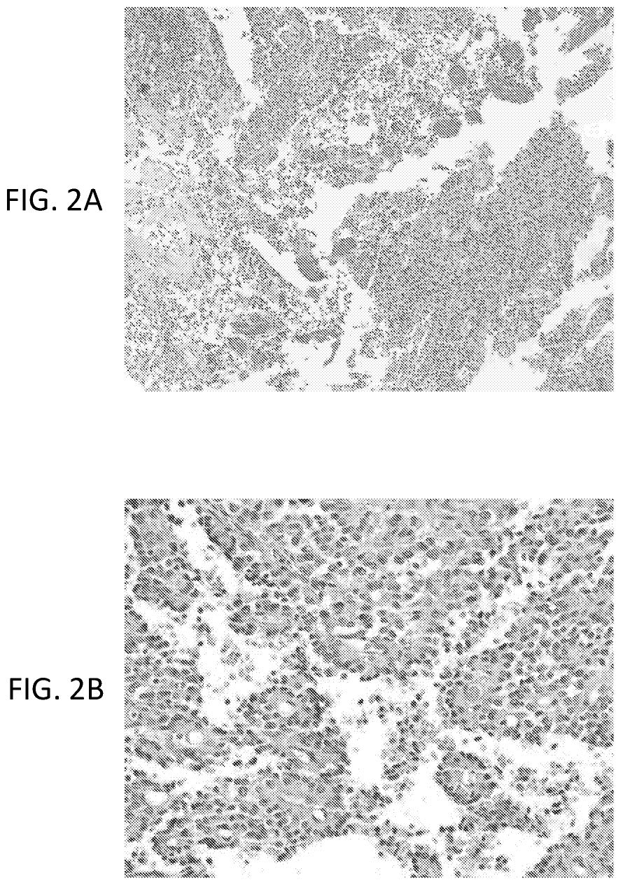

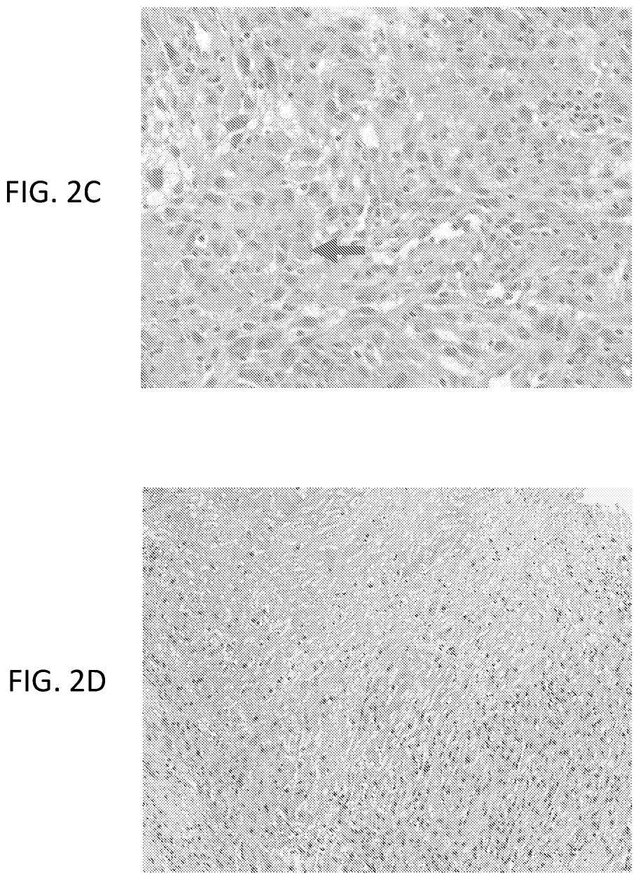

Among the 16 PBRM1-mutant cases (2.8% of cohort), the detected PBRM1 alterations included six intragenic deletions, four frame-shifting insertions, four frame-shifting deletions and two truncating mutations (FIG. 1). All showed biallelic inactivation by SNP array analysis and mutant allele read count data analysis. Median tumor mutational burden (TMB) was 2.1 mutations/Mb and all cases were microsatellite stable. Representative histopathology of PBRM1-mutant meningiomas are provided in FIGS. 2A-2D. The majority of PBRM1-mutant meningiomas occurred in female patients (n=10/16, 62.5%), and median age was 51 years. Most cases were located supratentorially (n=10). 10/16 cases were known recurrences. Additional characteristics of PBRM1-mutant meningiomas are shown in Table 1.

TABLE 1
Location, histology, and molecular characteristics of PBRM1-mutant meningioma

| | | | | Histologic | | PBRM1 | Concurrent | TMB |
|---|---|---|---|---|---|---|---|---|
| Patient | | Age | Tumor | subtype/WHO | PBRM1 | allele | Genomic | (mutations/ |
| No. | Gender | (years) | location | grade | mutation | frequency | Alterations | mB) |
| 1 | male | 55 | temporal | Papillary/III | p.F732fs*13 | 60 | NF2 p.M369fs*5, | 6.1 |
| | | | | | | | TBX3 | |
| | | | | | | | p.E384fs*22, two | |
| | | | | | | | copy loss of | |
| | | | | | | | CDKN2A | |
| 2 | female | 50 | adrenal gland | Papillary/III | Two copy | | CREBBP p.G57V, | 1.7 |
| | | | | | number loss | | two copy loss of | |
| | | | | | | | CDKN2A and | |
| | | | | | | | BAP1 | |
| 3 | female | 42 | cavernous | Papillary/III | p.R146* | 50.9 | BAP1 p.Q260* | 3.5 |
| | | | sinus | | | | | |
| 4 | female | 60 | left frontal | Papillary | p.A482fs*18 | 53.4 | None | 2.6 |
| | | | | features/I | | | | |
| 5 | male | 69 | left | Papillary | p.Q949fs*59 | 65 | None | 0.9 |
| | | | frontoparietal | features/II | | | | |
| 6 | female | 37 | left | Papillary | p.E1029fs*100 | 34 | None | <0.1 |
| | | | supraorbital | features/II | | | | |
| 7 | male | 42 | right | Papillary | p.K1372* | 13.9 | None | <0.1 |
| | | | supratentorial | features/II | | | | |
| 8 | male | 46 | frontoparietal | Papillary | Two copy | | NF2 p.R8fs*36, | 3.5 |
| | | | | features/II | number loss | | ASXL1 | |
| | | | | | | | p.G967del, two | |
| | | | | | | | copy loss of BAP1 | |
| 9 | female | 45 | right | Papillary | Two copy | | FBXW7 G419*, | 3.8 |
| | | | cerebellopontine | features/I | number loss | | NOTCH1 | |
| | | | angle | | | | V1575L, two | |
| | | | | | | | copy loss of BAP1 | |
| 10 | female | 67 | right | Papillary | Two copy | | Two copy loss of | 1.7 |
| | | | infratentorial | features/II | number loss | | BAP1 | |
| 11 | female | 51 | right | Papillary & | Two copy | | None | 2.6 |
| | | | frontoparietal | rhabdoid | number loss | | | |
| | | | | features/II | | | | |
| 12 | female | 71 | parasagittal | Anaplastic/III | p.S39fs*14 | 41 | NF2 p.K80*, | 0.9 |
| | | | | | | | PTEN p.D92E | |
| 13 | male | 70 | left frontal | Rhabdoid/III | p.S652fs*13 | 18 | SETD2 | 2.4 |
| | | | | | | | p.A862fs*2, VHL | |
| | | | | | | | p.W117C, HGF | |
| | | | | | | | amplification | |
| 14 | female | 58 | left neck | Rhabdoid/III | Two copy | | Two copy loss of | 4.8 |
| | | | | | number loss | | BAP1 | |
| 15 | female | 51 | right parieto- | Rhabdoid | p.L1565fs*31 | 55.5 | TP53 p.T211I, | <0.1 |
| | | | occipital | features/II | | | two copy loss of | |
| | | | | | | | BAP1 | |
| 16 | male | 52 | left cavernous | Chordoid/II | p.V964fs*18 | 7 | None | 1.2 |
| | | | sinus | | | | | |

A notable feature of the cohort was the frequent overlap of PBRM1 mutation with mutations in BAP1 (n=5). Three of these five cases displayed papillary features while two displayed rhabdoid features. An association between BAP1 mutation and rhabdoid histology has been previously described (Shankar, G. M. et al. Neuro Oncol. 2017 19:4; Shankar, G. M. & Santagata, S. Neuro Oncol. 2017 19:11). This association was confirmed in the cohort of 562 meningiomas, in which 13 of 17 cases that were BAP1-mutant/PBRM1-wt had rhabdoid features.

Among the 19 PBRM1-wt meningiomas with papillary histology, two had mutations in BAP1, consistent with prior reports of rare BAP1-mutant cases with papillary morphology (Shankar, G. M. et al. Neuro Oncol. 2017 19:4; Wadt, K. A. W. et al. Clin Genet. 2015 88:3). Notably, meningiomas that were BAP1-wt/PBRM1-mutant frequently had papillary morphology (7 of 11). Without wishing to be bound by theory, the present findings indicate that BAP1 mutations tend to occur in rhabdoid meningiomas whereas PBRM1 mutations tend to occur in papillary meningiomas, although genetic and histologic overlap is noted.

In conclusion, the tumor suppressor gene PBRM1 was identified as a recurrently altered gene in meningiomas with papillary histomorphology. The present findings therefore indicate a utility for PBRM1 mutations in meningiomas in research, diagnostic and clinical settings.

1. A method of treating or delaying progression of meningioma in an individual, comprising subjecting the individual to a therapy selected from the group consisting of aggressive tumor resection, an adjuvant therapy, an anti-cancer agent, a cancer immunotherapy, and combinations thereof, wherein the individual has an inactivating mutation of polybromo 1 (PBRM1).

2. The method of claim 1, wherein an inactivating mutation of PBRM1 has been detected in a sample from the individual prior to subjecting the individual to the therapy.

3. The method of claim 1, further comprising, prior to subjecting the individual to the therapy, detecting an inactivating mutation of PBRM1 in a sample from the individual.

4-44. (canceled)

45. The method of claim 2, wherein the presence of the inactivating mutation of PBRM1 is detected in DNA or RNA from the sample.

46. The method of claim 45, wherein the presence of the inactivating mutation of PBRM1 is detected by polymerase chain reaction (PCR), Sanger sequencing, next-generation sequencing (NGS), single nucleotide polymorphism (SNP) array, or fluorescence in situ hybridization (FISH).

47. The method of claim 2, wherein the presence of the inactivating mutation of PBRM1 is detected in protein from the sample.

48-59. (canceled)

60. The method of claim 1, wherein the inactivating mutation of PBRM1 is loss of a PBRM1 allele.

61-63. (canceled)

64. The method of claim 1, wherein the inactivating mutation of PBRM1 is selected from the group consisting of insertions, deletions, intragenic deletions, frame-shifting insertions, frame-shifting deletions, truncating mutations and splice site mutations.

65. The method of claim 64, wherein the inactivating mutation of PBRM1 is selected from the group consisting of F732fs*13, R146*, A482fs*18, Q949fs*59, E1029fs*100, K1372*, S39fs*14, S652fs*13, L1565fs*31 and V964fs*18.

66-71. (canceled)

72. The method of claim 2, wherein the sample is a whole blood, serum, plasma, bone marrow, cerebrospinal fluid (CSF), tumor, or tissue sample.

73. The method of claim 2, wherein the sample is from amniotic fluid, blood, plasma, serum, semen, lymphatic fluid, cerebral spinal fluid, ocular fluid, urine, saliva, stool, mucus, sweat, blood, skin, hair, hair follicles, saliva, oral mucous, vaginal mucus, sweat, tears, epithelial tissues, urine, semen, seminal fluid, seminal plasma, prostatic fluid, Cowper's fluid, excreta, biopsy, ascites, cerebrospinal fluid, or lymph.

74-75. (canceled)

76. The method of claim 2, wherein the sample comprises tumor nucleic acids.

77-83. (canceled)

84. The method of claim 3, further comprising detecting one or more additional mutations in the meningioma or the sample.

85. (canceled)

86. The method of claim 84, wherein the one or more additional mutations are in one or more genes selected from the group consisting of VF2, TBX3, CDKN2A, CREBBP, BAP1, NF2, ASXL1, FBXW7, NOTCH1 PTEN, SETD2, VHL, HGF, and TP53.

87. (canceled)

88. The method of claim 1, further comprising assessing histologic features of a tumor sample from the individual.

89. The method of claim 88, wherein the tumor does not have obvious papillary features.

90. The method of claim 88, wherein the tumor is papillary or has papillary features.

91. The method of claim 88, wherein the tumor is rhabdoid or has rhabdoid features.

92. The method of claim 88, wherein the tumor has heterogeneous histologic features.

93-94. (canceled)

95. The method of claim 1, wherein the individual is human.

96-104. (canceled)
